(19) United States
(12) Patent Application Publication
(10) Pub. No.: US 2023/0073101 A1
(43) Pub. Date: Mar. 9, 2023

(54) MICROALGAL EXTRACELLULAR POLYMERIC SUBSTANCES AND AGRICULTURAL USES THEREOF

(71) Applicant: HELIAE DEVELOPMENT, LLC, Gilbert, AZ (US)

(72) Inventors: Taner Bicer, Gilbert, AZ (US); Chiliang Chen, Ames, IA (US); Eric Lichtenheld, Gilbert, AZ (US); Paul Lim, Chandler, AZ (US); Rosaicela Roman Gomez, Gilbert, AZ (US); Christopher Tee, Chandler, AZ (US); Yan Xu, Tempe, AZ (US); Julian Yu, Phoenix, AZ (US); Erik Velkme, Tempe, AZ (US)

(21) Appl. No.: 17/816,921

(22) Filed: Aug. 2, 2022

Related U.S. Application Data
(60) Provisional application No. 63/228,585, filed on Aug. 2, 2021.

(51) Int. Cl. A01N 65/03; A01N 63/20; C12N 1/12

(52) U.S. Cl. CPC A01N 65/03; A01N 63/20; C12N 1/125

(57) ABSTRACT

The present disclosure provides a composition comprising: an extracellular polymeric substance produced by microalgae; plant growth promoting Gram-negative bacteria; and an agriculturally acceptable carrier. Also provided are an isolated biologically pure culture of Parachlorella kessleri Accession No. NCMA 202103001, a mutant thereof having all the identifying characteristics thereof, or a cell-free preparation or extracellular polymeric substance thereof, which may be used for plant enhancement and improving health of soil.

### CROSS REFERENCE TO RELATED APPLICATIONS

This application claims priority to U.S. Provisional Patent Application No. 63/228,585, filed Aug. 2, 2021, entitled MICROALGAL EXTRACELLULAR POLYMERIC SUBSTANCES AND AGRICULTURAL USES THEREOF, the entire content of which is hereby incorporated by reference herein.

### STATEMENT OF GOVERNMENT SUPPORT

This invention was made with government support under Grant No. DE-SC0015662 awarded by the U.S. Department of Energy. The government has certain rights in the invention.

### STATEMENT REGARDING ELECTRONIC FILING OF A SEQUENCE LISTING

The contents of the electronic sequence listing filed herewith, entitled, “069A4HO210802US_Sequence_Listing_20220802.xml” (Size: 6,073 bytes; Date of Creation: Aug. 2, 2022), is herein incorporated by reference in its entirety.

### TECHNICAL FIELD

The present invention relates to microalgal compositions and methods useful in agriculture for enhancing plant characteristics and improving soil health.

### BACKGROUND

It is a common practice in the agricultural field both for food production, ornamental shrubs and trees, and lawn grasses to accelerate growth by the application of chemical fertilizers, e.g., nitrates, phosphates, and potassium compounds, and chemical materials such as pesticides, herbicides, and fungicides, etc. Further, it is a present practice to overload the crops with these chemical materials and to repeatedly treat most crops multiple times in a growing season (typically four times and as many as eight times depending on the plant and location) because these water-soluble substances would wash away from irrigation and rainfall events. The significant amount of runoff means that growers must use more of these substances and apply more times, which increases both the monetary and labor cost. The runoff also results in these chemical materials finding their way into the soil and the ground water, and into rivers, lakes, ponds and ultimately the bays and oceans. While these chemicals do enhance the growth of desirable plants, the runoff often has toxic effects. Thus, there is a need for environmentally friendly and sustainable means for enhancing plant growth and improving soil health.

It has now been recognized that various characteristics including the quality, health, and/or color of plants can be improved through the application of effective amounts of biomass, extracts, and other biological products obtained from the cell tissue of microalgal species. In addition, application of certain of these microalgal substances to soil can have a beneficial impact on the various aspects of the soil including the soil microbiome, soil aggregation, and water retention thereby providing a more productive growth medium for plants. Soils with an improved microbiome and aggregates tend to have less runoff and to keep materials on the field thereby reducing the need for repeat application of chemical fertilizers, pesticides, and other agrochemicals. There is a need to develop effective agricultural microalgal products to supplement or replace agrochemicals and improve soil health, crop growth, and yield in a sustainable manner.

### SUMMARY

The present disclosure relates to a composition comprising: an extracellular polymeric substance produced by microalgae; plant growth promoting Gram-negative bacteria; and an agriculturally acceptable carrier. In one aspect, the composition is an agricultural composition.

In some aspects, the composition further comprises biomass produced by the microalgae or a fraction thereof. In one aspect, the fraction is proteins. In another aspect, the fraction is lipids. In another aspect, the fraction is carbohydrates. In one aspect, the carbohydrates comprise disaccharides including but not limited to trehalose and sucrose.

In certain aspects, the biomass is a whole broth culture or a cell pellet of the microalgae.

In one aspect, the extracellular polymeric substance and/or the biomass or fraction thereof increase the resistance to desiccation of the plant growth promoting Gram-negative bacteria compared to that of the plant growth promoting Gram-negative bacteria alone.

In other aspects, the plant growth promoting Gram-negative bacteria belong to a genus selected from the group consisting of Pseudomonas, Burkholderia, Stenotrophomonas, Rhizobium, Bradyrhizobium, Sinorhizobium, Azospirillum, Herbaspirillum, Lysobacter, Pantoea, Azotobacter, Enterobacter, Klebsiella, Kosakonia, Rahnella, Sphingomonas, Massilia, Gluconacetobacter, Acetobacter, Asaia, Komagataeibacter, Nguyenibacter, Swaminathania, Janthinobacterium, Duganella, Methylobacterium, Flavobacterium, Serratia, Variovorax, and combinations thereof.

In some aspects, the microalgae are green algae in the order Chlorellales. In one aspect, the green algae belong to a genus selected from the group consisting of Acanthosphaera, Actinastrum, Apatococcus, Apodococcus, Auxenochlorella, Catena, Chlorella, Chloroparva, Closteriopsis, Compactochlorella, Coronastrum, Cylindrocelis, Diacanthos, Dicellula, Dicloster, Dictyosphaerium, Didymogenes, Dunaliella, Fissuricella, Follicularia, Geminella, Gloeotila, Golenkiniopsis, Hegewaldia, Helicosporidium, Heynigia, Hindakia, Hormospora, Kalenjinia, Keratococcus, Leptochlorella, Marasphaerium, Marinichlorella, Marvania, Masaia, Meyerella, Micractinium, Mucidosphaerium, Muriella, Nannochloris, Nanochlorum, Palmellochaete, Parachlorella, Planktochlorella, Podohedra, Prototheca, Pseudochloris, Pseudosiderocelopsis, Pumiliosphaera, Scenedesmus, Siderocelis, and Zoochlorella.

In another aspect, the green algae belong to the genus Parachlorella. In some aspects, the green algae belong to one or more of the following species: Parachlorella kessleri, Parachlorella beijerinckii, and Parachlorella hussii. In one aspect, the green algae belong to the species Parachlorella kessleri. In another aspect, the green algae are Parachlorella kessleri Accession No. NCMA 202103001.

In another aspect, the green algae belong to the genus Chlorella. In some aspects, the green algae belong to one or more of the following species: Chlorella anitrata, Chlorella autotrophica, Chlorella colonials, Chlorella lewinii, Chlorella minutissima, Chlorella pituita, Chlorella protothecoides, Chlorella pulchelloides, Chlorella pyrenoidosa, Chlorella rotunda, Chlorella saccharophila, Chlorella singularis, Chlorella sorokiniana, Chlorella variabilis, Chlorella volutis, and Chlorella vulgaris. In one aspect, the green algae belong to the species Chlorella vulgaris.

In another aspect, the green algae belong to the genus Micractinium. In some aspects, the green algae belong to one or more of the following species: Micractinium bornhemiense, Micractinium inermum, Micractinium pusillum, and Micractinium quadrisetum. In one aspect, the green algae belong to the species Micractinium inermum.

In another aspect, the green algae belong to the genus Scenedesmus. In some aspects, the green algae belong to one or more of the following species: Scenedesmus abundans, Scenedesmus acuminatus, Scenedesmus acutus, Scenedesmus acutus alternans, Scenedesmus bicaudatus, Scenedesmus bijuga, Scenedesmus bijuga alternans, Scenedesmus braziliensis, Scenedesmus denticulatus, Scenedesmus dimorphus, Scenedesmus incrassatulus, Scenedesmus obliquus, Scenedesmus quadricauda, Scenedesmus quadrispina, and Scenedesmus serratus. In one aspect, the green algae belong to the species Scenedesmus obliquus.

In yet other aspects, the microalgae are red algae in the phylum Rhodophyta. Examples of red algae include but are not limited to Gelidium amansii, Cottonii, Grateloupia lanceolata, Galdieria sulphuraria, Porphyra suborbiculata, Porphyridium purpureum, Porphyridium cruentum, Pterocladia tenuis, Acanthopeltis japonica, Gloiopeltis tenax, Gracilaria verrucosa, Chondrus ocelatus, Pachymeniopsis elliptica, Hypnea charoides, Ceramium kondoi, Ceramium boydenii, Galdieria phlegrea, Gigartina tenella, Campylaephora hypnaeoides, Grateloupia filicina, and Rhodella articulata.

In another aspect, the red algae belong to the genus Porphyridium. In some aspects, the red algae belong to one or more of the following species: Porphyridium aerugineum, Porphyridium purpureum, Porphyridium sordidum, Porphyridium cruentum, Porphyridium griseum, Porphyridium marinum, Porphyridium schinzii, Porphyridium violaceum, and Porphyridium wittrockii. In one aspect, the red algae belong to the species of Porphyridium cruentum.

In another aspect, the red algae belong to the genus Rhodella. In some aspects, the green algae belong to one or more of the following species: Rhodella cyanea, Rhodella grisea, Rhodella maculate, Rhodella purpureum, Rhodella reticulata, and Rhodella violaceae. In one aspect, the red algae belong to the species of Rhodella reticulata.

In some aspects, the red algae belong to a genus selected from the group consisting of Acanthopeltis, Campylaephora, Ceramium, Chondrus, Hypnea, Galdieria, Gelidium, Gigartina, Gloiopeltis, Gracilaria, Grateloupia, Pachymeniopsis, Porphyra, Porphyridium, Pterocladia, and Rhodella. In one aspect, the red algae belong to the genus Porphyridium. In another aspect, the red algae are Porphyridium cruentum. In another aspect, the red algae are Porphyridium cruentum UTEX 161.

In certain aspects, the extracellular polymeric substance comprises an exopolysaccharide. In other aspects, the extracellular polymeric substance comprises an exopolysaccharide and protein. In one aspect, the exopolysaccharide has a molecular weight of between about 100 kD and about 150 kD. In another aspect, the exopolysaccharide has a molecular weight of between about 110 kD and about 140 kD. In another aspect, the exopolysaccharide has a molecular weight of about 120 kD.

In some aspects, at least 45%, at least 50%, or at least 55% of the monosaccharides in the exopolysaccharide are galactose. In other aspects, about 50% to about 70% of the monosaccharides in the exopolysaccharide are galactose. In one aspect, about 55% to about 65% of the monosaccharides in the exopolysaccharide are galactose.

In other aspects, at least 1%, at least 5%, or at least 10% of the monosaccharides in the exopolysaccharide are rhamnose. In other aspects, about 1% to about 20% of the monosaccharides in the exopolysaccharide are rhamnose. In one aspect, about 7% to about 17% of the monosaccharides in the exopolysaccharide are rhamnose.

In other aspects, at least 1%, at least 5%, or at least 8% of the monosaccharides in the exopolysaccharide are mannose. In other aspects, about 1% to about 20% of the monosaccharides in the exopolysaccharide are mannose. In one aspect, about 5% to about 15% of the monosaccharides in the exopolysaccharide are mannose.

In other aspects, at least 1%, at least 3%, or at least 6% of the monosaccharides in the exopolysaccharide are xylose. In other aspects, about 1% to about 15% of the monosaccharides in the exopolysaccharide are xylose. In one aspect, about 3% to about 13% of the monosaccharides in the exopolysaccharide are xylose.

In other aspects, at least 1% or at least 3% of the monosaccharides in the exopolysaccharide are glucose. In other aspects, about 1% to about 15% of the monosaccharides in the exopolysaccharide are glucose. In one aspect, about 1% to about 10% of the monosaccharides in the exopolysaccharide are glucose.

In other aspects, at least 1% or at least 2% of the monosaccharides in the exopolysaccharide are glucuronic acid. In other aspects, about 1% to about 10% of the monosaccharides in the exopolysaccharide are glucuronic acid. In one aspect, about 1% to about 5% of the monosaccharides in the exopolysaccharide are glucuronic acid.

In other aspects, at least 1% or at least 2% of the monosaccharides in the exopolysaccharide are arabinose. In other aspects, about 1% to about 10% of the monosaccharides in the exopolysaccharide are arabinose. In one aspect, about 1% to about 5% of the monosaccharides in the exopolysaccharide are arabinose.

In one aspect, the exopolysaccharide comprises the monosaccharides identified in Table 3. In another aspect, the exopolysaccharide comprises the glycosyl linkages identified in Table 4.

In some aspects, the extracellular polymeric substance has a viscosity of between about 130000 mPa·s and about 150000 mPa·s at a shear rate of 1 s⁻¹ and/or a viscosity of between about 40 mPa·s and about 60 mPa·s at a shear rate of 1000 s⁻¹. In one aspect, the extracellular polymeric substance has a viscosity of between about 132000 mPa·s and about 142000 mPa·s at a shear rate of 1 s⁻¹. In another aspect, the extracellular polymeric substance has a viscosity of between about 42 mPa·s and about 52 mPa·s at a shear rate of 1000 s⁻¹.

In other aspects, the extracellular polymeric substance has a zero shear viscosity of between about 400000 mPa·s and about 440000 mPa·s. In one aspect, the extracellular polymeric substance has a zero shear viscosity of between about 405000 mPa·s and about 435000 mPa·s.

In certain aspects, the extracellular polymeric substance has a complex modulus plateau of between about 20 Pa and about 50 Pa and/or a phase angle plateau of between about 20° and about 30°. In other aspects, the extracellular polymeric substance has a complex modulus plateau of between about 30 Pa and about 40 Pa. In yet other aspects, the extracellular polymeric substance has a phase angle plateau of between about 22° and about 28°.

In certain aspects, the compositions disclosed herein are formulated as a seed treatment.

In one aspect, the plant growth promoting Gram-negative bacteria are present in the composition as an isolated biologically pure culture.

In other aspects, the present invention relates to a plant propagation material treated with a composition disclosed herein. In one aspect, the plant propagation material is a seed.

In some aspects, the present disclosure is directed to a method of increasing resistance to desiccation in plant growth promoting Gram-negative bacteria, the method comprising adding an extracellular polymeric substance produced by microalgae to the plant growth promoting Gram-negative bacteria.

In some aspects, the method further comprises adding biomass produced by the microalgae or a fraction thereof to the plant growth promoting Gram-negative bacteria.

In one aspect, the plant growth promoting Gram-negative bacteria are present as an isolated biologically pure culture upon addition of the extracellular polymeric substance produced by microalgae.

In other aspects, the present disclosure provides an isolated biologically pure culture of Parachlorella kessleri Accession No. NCMA 202103001 or a mutant thereof having all the identifying characteristics thereof. In one aspect, the present disclosure relates to a cell-free preparation or extracellular polymeric substance of the isolated biologically pure culture of Parachlorella kessleri Accession No. NCMA 202103001 or mutant thereof.

In yet other aspects, the present disclosure provides a composition comprising an isolated biologically pure culture of Parachlorella kessleri Accession No. NCMA 202103001, a mutant thereof having all the identifying characteristics thereof, or a cell-free preparation or extracellular polymeric substance thereof; and an agriculturally acceptable carrier.

In one aspect, the present disclosure provides a plant propagation material treated with a composition comprising an isolated biologically pure culture of Parachlorella kessleri Accession No. NCMA 202103001, a mutant thereof having all the identifying characteristics thereof, or a cell-free preparation or extracellular polymeric substance thereof.

In certain aspects, the present disclosure relates to a method of plant enhancement comprising the step of: applying to a plant, a plant part and/or a plant locus an effective amount of an isolated biologically pure culture of Parachlorella kessleri Accession No. NCMA 202103001, a mutant thereof having all the identifying characteristics thereof, or a cell-free preparation or extracellular polymeric substance thereof to enhance at least one plant characteristic.

In one aspect, the isolated biologically pure culture of Parachlorella kessleri Accession No. NCMA 202103001 or mutant thereof comprises whole cells, lysed cells, or a combination thereof.

In another aspect, the present disclosure relates to a seed treatment comprising a composition comprising a biologically pure culture of Parachlorella kessleri Accession No. NCMA 202103001 or a mutant thereof or a cell-free preparation or extracellular polymeric substance of Parachlorella kessleri Accession No. NCMA 202103001 or a mutant thereof.

In some aspects, the at least one plant characteristic is selected from the group consisting of seed germination rate, seed germination time, seedling emergence, seedling emergence time, seedling size, plant fresh weight, plant dry weight, utilization, fruit production, leaf production, leaf formation, leaf size, leaf area index, plant height, thatch height, plant health, plant resistance to salt stress, plant resistance to heat stress, plant resistance to heavy metal stress, plant resistance to drought, maturation time, yield, root length, root mass, color, blossom end rot, softness, plant quality, fruit quality, flowering, sunburn, and any combination thereof. In one aspect, the plant characteristic is plant fresh weight, plant dry weight, or yield.

In some aspects, the plant is a member of a plant family selected from: Solanaceae, Fabaceae (Leguminosae), Poaceae, Roasaceae, Vitaceae, Brassicaeae (Cruciferae), Caricaceae, Malvaceae, Sapindaceae, Anacardiaceae, Rutaceae, Moraceae, Convolvulaceae, Lamiaceae, Verbenaceae, Pedaliaceae, Asteraceae (Compositae), Apiaceae (Umbelliferae), Araliaceae, Oleaceae, Ericaceae, Actinidaceae, Cactaceae, Chenopodiaceae, Polygonaceae, Theaceae, Lecythidaceae, Rubiaceae, Papveraceae, Illiciaceae Grossulariaceae, Myrtaceae, Juglandaceae, Bertulaceae, Cucurbitaceae, Asparagaceae (Liliaceae), Alliaceae (Liliceae), Bromeliaceae, Zingieraceae, Muscaceae, Areaceae, Dioscoreaceae, Myristicaceae, Annonaceae, Euphorbiaceae, Lauraceae, Piperaceae, and Proteaceae.

In certain aspects, the present disclosure relates to a method of improving health of soil comprising the step of administering to the soil a composition comprising an isolated biologically pure culture of Parachlorella kessleri Accession No. NCMA 202103001, a mutant thereof having all the identifying characteristics thereof, or a cell-free preparation or extracellular polymeric substance thereof in an effective amount to at least one of: increase an amount of active carbon in the soil, increase an amount of protein in the soil, increase an amount of culturable bacteria in the soil, decrease an amount of total suspended solids lost in run-off from the soil, decrease an amount of total dissolved solids lost in run-off from the soil, increase water holding capacity of the soil, and increase soil aggregation.

In other aspects, the present disclosure relates to a method of improving health of soil comprising the step of administering to the soil a composition comprising an isolated biologically pure culture of Parachlorella kessleri Accession No. NCMA 202103001, a mutant thereof having all the identifying characteristics thereof, or a cell-free preparation or extracellular polymeric substance thereof in an effective amount to at least one of: increase an amount of active carbon in the soil, increase an amount of protein in the soil, increase an amount of culturable bacteria in the soil, decrease in soil crusting, decrease in soil compaction, decrease an amount of total suspended solids lost in run-off from the soil, decrease an amount of total dissolved solids lost in run-off from the soil, increase water holding capacity of the soil, and increase soil aggregation.

In one aspect, soil aggregation is increased in the treated soil compared to substantially identical untreated soil. In another aspect, water holding capacity is increased in the treated soil compared to substantially identical untreated soil. In another aspect, culturable bacteria are increased in the treated soil compared to substantially identical untreated soil.

In certain aspects, the soil is loam soil, sandy loam soil, or sand soil. In one aspect, the soil is sand soil.

In other aspects, the compositions disclosed herein are formulated as a solid, liquid, or gel. In one aspect, the solid formulation is selected from the group consisting of a powder, lyophilizate, pellet, and granule. In another aspect, the liquid formulation is selected from the group consisting of an emulsion, colloid, suspension, and solution.

In certain aspects, the compositions disclosed herein further comprise at least one culture stabilizer selected from the group consisting of potassium sorbate, phosphoric acid, ascorbic acid, sodium benzoate, or a combination thereof.

In some aspects, the composition is applied as a soil drench, an in-furrow treatment, a foliar application, a side-dress application, a pivot irrigation application, a seed coating, or with a drip system.

In other aspects, the composition is administered at a rate of 0.1-150 gallons per acre (0.935-1402.5 liters per hectare).

### BRIEF DESCRIPTION OF THE DRAWINGS

FIG. 1 depicts the colony morphology of 1) a Parachlorella kessleri wild-type strain; and 2) Parachlorella kessleri Accession No. NCMA 202103001.

FIG. 2 depicts size exclusion chromatograms with EPS from Parachlorella kessleri Accession No. NCMA 202103001 detected by refractive index (RI) (top) or ultraviolet (UV) absorbance at 280 nm (bottom).

FIG. 3 depicts size exclusion chromatograms with molecular weight standards using RI detection.

FIG. 4 depicts a gas chromatography/mass spectrometry (GC/MS) chromatogram of the per-O-trimethylsilyl (TMS) methyl glycosides generated with Parachlorella kessleri Accession No. NCMA 202103001 EPS.

FIG. 5 depicts a pie chart with the relative percentages of carbohydrate residues present in the Parachlorella kessleri Accession No. NCMA 202103001 EPS.

FIG. 6 depicts a GC/MS chromatogram of the partially methylated alditol acetate (PMAA) derivatives of the Parachlorella kessleri Accession No. NCMA 202103001 EPS used to identify glycosyl linkages.

FIG. 7 depicts the measurement of viscosity (Pa·s) versus shear rate (1/s) with Parachlorella kessleri Accession No. NCMA 202103001 EPS.

FIG. 8 depicts the measurement of viscosity (Pa·s) versus stress (Pa) with Parachlorella kessleri Accession No. NCMA 202103001 EPS.

FIG. 9 depicts the measurement of complex modulus (Pa) versus oscillation stress (Pa) with Parachlorella kessleri Accession No. NCMA 202103001 EPS.

FIG. 10 depicts the measurement of phase angle)(° versus oscillation stress (Pa) with Parachlorella kessleri Accession No. NCMA 202103001 EPS.

FIG. 11 depicts the measurement of viscosity (Pa·s) versus stress (Pa) with Parachlorella kessleri Accession No. NCMA 202103001 EPS, Porphyridium cruentum UTEX 161 EPS, and xanthan gum.

FIG. 12 depicts the measurement of complex modulus (Pa) versus oscillation stress (Pa) with Parachlorella kessleri Accession No. NCMA 202103001 EPS, Porphyridium cruentum UTEX 161 EPS, and xanthan gum.

FIG. 13 depicts the measurement of phase angle)(° versus oscillation stress (Pa) with Parachlorella kessleri Accession No. NCMA 202103001 EPS, Porphyridium cruentum UTEX 161 EPS, and xanthan gum.

FIG. 14 depicts the measurement of soil water holding capacity in sand soil from Douglas, Ga. treated with 1) live-cell crude biomass; 2) live-cell washed biomass; 3) supernatant; 4) lysed crude biomass; 5) lysed washed biomass; 6) PHYCOTERRA® (whole cell Chlorella microalgae); and 7) untreated control (“UTC”). Treatments 1) to 5) were each with Parachlorella kessleri Accession No. NCMA 202103001.

FIG. 15 depicts the measurement of dry soil aggregate size in sand soil from Douglas, Ga. treated with 1) live-cell crude biomass; 2) live-cell washed biomass; 3) supernatant; 4) lysed crude biomass; 5) lysed washed biomass; 6) PHYCOTERRA® (whole cell Chlorella microalgae); and 7) UTC. Treatments 1) to 5) were each with Parachlorella kessleri Accession No. NCMA 202103001.

FIG. 16 depicts the measurement of culturable bacterial populations in sand soil from Douglas, Ga. treated with 1) live-cell crude biomass; 2) live-cell washed biomass; 3) supernatant; 4) lysed crude biomass; 5) lysed washed biomass; 6) PHYCOTERRA® (whole cell Chlorella microalgae); and 7) UTC. Treatments 1) to 5) were each with Parachlorella kessleri Accession No. NCMA 202103001.

FIG. 17 depicts a principal coordinate analysis (PCoA) based on Bray-Curtis distance of soil samples from Douglas, Ga. grouped by the following treatments: 1) live-cell crude biomass; 2) live-cell washed biomass; 3) supernatant; 4) lysed crude biomass; 5) lysed washed biomass; 6) PHYCOTERRA® (whole cell Chlorella microalgae) (“PT”); and 7) UTC. Treatments 1) to 5) were each with Parachlorella kessleri Accession No. NCMA 202103001.

FIG. 18 depicts a PCoA based on Bray-Curtis distance of soil samples from Granger, Iowa grouped by the following treatments: 1) live-cell crude biomass; 2) live-cell washed biomass; 3) supernatant; 4) lysed crude biomass; 5) lysed washed biomass; 6) PHYCOTERRA® (whole cell Chlorella microalgae) (“PT”); and 7) UTC. Treatments 1) to 5) were each with Parachlorella kessleri Accession No. NCMA 202103001.

FIG. 19 depicts a summary of the first 20 most abundant operational taxonomic units (OTUs) according to soil source and treatment. Analyses of soil samples from Douglas, Ga. and Granger, Iowa are shown. Soil samples were treated with 1) live-cell crude biomass; 2) live-cell washed biomass; 3) supernatant; 4) lysed crude biomass; 5) lysed washed biomass; 6) PHYCOTERRA® (whole cell Chlorella microalgae) (“PT”); and 7) UTC. Treatments 1) to 5) were each with Parachlorella kessleri Accession No. NCMA 202103001. The different shades of bars represent distinct genera in the phyla indicated along the x-axis.

FIG. 20 depicts the measurement of desiccation resistance with Kosakonia pseudosacchari strain C19-Rif mixed with Parachlorella kessleri Accession No. NCMA 202103001 EPS (“Algal EPS”, crude EPS with cellular materials) alone or in combination with bacterial EPS (supernatant without cellular materials).

FIGS. 21A and 21B depict the average colony forming units (CFU) on a log scale of cultured Kosakonia pseudosacchari strain C19-Rif cells (at an initial cell density of 2×10⁹ CFU/mL) recovered after being treated with water (untreated control or “UTC”), 60% glycerol (i.e., at a high concentration to create glycerol stress), Porphyridium cruentum UTEX 161 whole broth (“Porphy Broth”), Porphyridium cruentum UTEX 161 supernatant (“Porphy Super”), Porphyridium cruentum UTEX 161 pellet (“Porphy Pellet”), Parachlorella kessleri Accession No. NCMA 202103001 whole broth (“3001 Broth”), Parachlorella kessleri Accession No. NCMA 202103001 supernatant (“3001 Super”), or Parachlorella kessleri Accession No. NCMA 202103001 Pellet (“3001 Pellet”) after 3 days of desiccation. FIG. 21A depicts the results when the protectants were added undiluted (i.e., at 1× dilution), while 21B depicts the results when the protectants were added at a dilution of 2× (i.e., at 2 parts water to 1 part protectant). The total viable cells were normalized to CFU per well of the 96-well plate on which the desiccation occurred (Log CFU/well).

FIGS. 22A, 22B, and 22C depict representative plates containing Kosakonia pseudosacchari strain C19-Rif cell colonies recovered after being diluted at various dilutions after 3 days of desiccation that were used to determine the values presented in FIGS. 21A and 21B.

FIGS. 23A and 23B depict the average colony forming units (CFU) on a log scale of cultured Kosakonia pseudosacchari strain C19-Rif cells (at an initial cell density of 2×10⁹ CFU/mL) recovered after being treated with water (untreated control or “UTC”), 60% glycerol (as a stressor), Porphyridium cruentum UTEX 161 whole broth (“Porphy Broth”), Porphyridium cruentum UTEX 161 supernatant (“Porphy Super”), Porphyridium cruentum UTEX 161 pellet (“Porphy Pellet”), Parachlorella kessleri Accession No. NCMA 202103001 whole broth (“3001 Broth”), Parachlorella kessleri Accession No. NCMA 202103001 supernatant (“3001 Super”), or Parachlorella kessleri Accession No. NCMA 202103001 Pellet (“3001 Pellet”) after 5 days of desiccation. FIG. 23A depicts the results when the protectants were added undiluted (i.e., at 1× dilution), while 23B depicts the results when the protectants were added at a dilution of 2×(i.e., at 2 parts water to 1 part protectant). The total viable cells were normalized to CFU per well of the 96-well plate on which the desiccation occurred (Log CFU/well).

FIGS. 24A, 24B, and 24C depict representative plates containing Kosakonia pseudosacchari strain C19-Rif cell colonies recovered after being diluted at various dilutions after 3 days of desiccation that were used to determine the values presented in FIGS. 23A and 23B.

FIG. 25A depicts the average colony forming units (CFU) on a log scale of cultured Kosakonia pseudosacchari strain C19-Rif cells (at an initial cell density of 1×10⁸ CFU/mL) recovered after being treated with water (untreated control or “UTC”), Porphyridium cruentum UTEX 161 whole broth (“Porphy Broth”), Porphyridium cruentum UTEX 161 supernatant (“Porphy Super”), and Porphyridium cruentum UTEX 161 pellet (“Porphy Pellet”), after 5 days of desiccation. The protectants were added either as undiluted (i.e., at 1× dilution), or at a dilution of 2×(i.e., at 2 parts water to 1 part protectant). The total viable cells were normalized to CFU per well of the 96-well plate on which the desiccation occurred (Log CFU/well). FIG. 25B depicts representative plates containing Kosakonia pseudosacchari strain C19-Rif cell colonies recovered after being diluted at 100× dilutions (showed as 10⁻² dilution) after 5 days of desiccation used to determine the values presented in FIG. 25A.

FIG. 26 depicts the average colony forming units (CFU) recovered using two different plating methods: a spot-plating method in which aliquots of 5 μL of each diluent were introduced as spots onto agar plates; or a spread-plating method in which a total of 100 μL of each diluent was transferred to the center of an agar plate before being spread (via several sterile glass beads) over all the surface of the plate. The total viable cells were normalized to the CFU per well of the 96-well plate on which the desiccation occurred (Log CFU/well).

### DETAILED DESCRIPTION

As used herein and in the appended claims, the singular forms “a,” “an,” and “the” include plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “microalgae” as used herein refers to microscopic single cell organisms such as microalgae, cyanobacteria, algae, diatoms, dinoflagellates, freshwater organisms, marine organisms, or other similar single cell organisms capable of growth in phototrophic, mixotrophic, or heterotrophic culture conditions.

The term “biomass” as used herein refers to the mass of biological materials produced by living organisms (e.g., algae, bacteria, etc.). The biomass includes, but is not limited to, living and dead cells and biological compounds produced by the cells including carbohydrates, proteins, and lipids.

As used herein, a “biologically pure” strain is intended to mean the strain separated from materials with which it is normally associated in nature. A strain associated with other strains, or with compounds or materials that it is not normally found with in nature, is still defined as “biologically pure.” A monoculture of a particular strain is, of course, “biologically pure.” In different embodiments, a “biologically pure” culture has been purified at least 2× or 5× or 10× or 50× or 100× or 1000× or higher (to the extent considered feasible by a skilled person in the art) from the material with which it is normally associated in nature. As a non-limiting example, if a culture is normally associated with soil, the organism can be biologically pure to an extent that its concentration in a given quantity of purified or partially purified material with which it is normally associated (e.g. soil) is at least 2× or 5× or 10× or 50× or 100× or 1000× or higher (to the extent considered feasible by a skilled person in the art) that in the original unpurified material.

The term “plant propagation material” is to be understood to denote all the generative parts of the plant such as seeds and vegetative plant material such as cuttings and tubers (e. g. potatoes), which can be used for the multiplication of the plant. This includes seeds, roots, fruits, tubers, bulbs, rhizomes, shoots, sprouts and other parts of plants, including seedlings and young plants, which are to be transplanted after germination or after emergence from soil.

As used herein, the term “agriculturally acceptable carrier” refers to a material that can be used to deliver an agriculturally beneficial agent to a plant, plant part or plant growth medium (e.g., soil). As used herein, the term “soil-compatible carrier” refers to a material that can be added to a soil without causing/having an unduly adverse effect on plant growth, soil structure, soil drainage, or the like. As used herein, the term “seed-compatible carrier” refers to a material that can be added to a seed without causing/having an unduly adverse effect on the seed, the plant that grows from the seed, seed germination, or the like. As used herein, the term “foliar-compatible carrier” refers to a material that can be added to a plant or plant part without causing/having an unduly adverse effect on the plant, plant part, plant growth, plant health, or the like.

By artificially controlling aspects of the microalgae culturing process such as the organic carbon feed (e.g., acetic acid, acetate), oxygen levels, pH, and light, the culturing process differs from the culturing process that microalgae experiences in nature. In addition to controlling various aspects of the culturing process, intervention by human operators or automated systems occurs during the culturing of microalgae through contamination control methods to prevent the microalgae from being overrun and outcompeted by contaminating organisms (e.g., fungi, bacteria). By intervening in the microalgae culturing process, the impact of the contaminating microorganisms can be mitigated by suppressing the proliferation of containing organism populations and the effect on the microalgal cells (e.g., lysing, infection, death, clumping). Thus, through artificial control of aspects of the culturing process and intervening in the culturing process with contamination control methods, the microalgae culture produced as a whole and used in the described inventive compositions differs from the culture that results from a microalgae culturing process that occurs in nature.

Certain publicly available strains described herein are identified by the term “UTEX” followed by a unique identifier containing letters and/or numbers. The term “UTEX” refers to the UTEX Culture Collection of Algae located at 205 W. 24th St., Biological Labs 218, The University of Texas at Austin (A6700), Austin, Tex. 78712 USA. The UTEX Culture Collection of Algae provides over 3,000 different strains of algae, representing more than 500 genera, to the public for a modest charge including the strains disclosed herein.

### Combinations of Gram-Negative Bacteria and Microalgal EPS

In certain aspects, the compositions of the present disclosure comprise an extracellular polymeric substance produced by microalgae; a plant growth promoting Gram-negative bacteria; and an agriculturally acceptable carrier. In some aspects, the compositions further comprise biomass or a fraction thereof produced by the microalgae. In one aspect, the microalgae are green algae. In another aspect, the microalgae are red algae.

Examples of genera of plant growth promoting Gram-negative bacteria include but are not limited to Pseudomonas, Burkholderia, Stenotrophomonas, Rhizobium, Bradyrhizobium, Sinorhizobium, Azospirillum, Herbaspirillum, Lysobacter, Pantoea, Azotobacter, Enterobacter, Klebsiella, Kosakonia, Rahnella, Sphingomonas, Massilia, Gluconacetobacter, Acetobacter, Asaia, Komagataeibacter, Nguyenibacter, Swaminathania, Janthinobacterium, Duganella, Methylobacterium, Flavobacterium, Serratia, and Variovorax.

In certain aspects, the plant growth promoting Gram-negative bacteria belong to the genus Pseudomonas. In one aspect, the plant growth promoting Gram-negative bacteria belong to one or more of the following species: Pseudomonas stutzeri, Pseudomonas fluorescens, Pseudomonas brassicacearum, Pseudomoas frederiksbergensis, Pseudomonas fulva, Pseudomonas syringae, Pseudomonas putida, Pseudomonas plecoglossicida, Pseudomonas mosselii, Pseudomonas gessardii, Pseudomonas libanensis, Pseudomonas oryzihabitans, and Pseudomonas geniculate. In another aspect, the plant growth promoting Gram-negative bacteria are Pseudomonas jessenii PS06.

In certain aspects, the plant growth promoting Gram-negative bacteria belong to the genus Kosakonia. In one aspect, the plant growth promoting Gram-negative bacteria belong to one or more of the following species: Kosakonia radicincitans, Kosakonia pseudosacchari, and Kosakonia sacchari. In another aspect, the plant growth promoting Gram-negative bacteria are one or more of: Kosakonia sacchari isolate PBC6, Kosakonia sacchari NCMA Accession No. 201701001, Kosakonia sacchari NCMA Accession No. 201701002, Kosakonia sacchari NCMA Accession No. 201701003, Kosakonia sacchari NCMA Accession No. 201701004, Kosakonia sacchari NCMA Accession No. 201708002, Kosakonia sacchari NCMA Accession No. 201708003, Kosakonia sacchari NCMA Accession No. 201708004, Kosakonia radicincitans NRRL Accession No. NRRL B-67171, and Kosakonia radicincitans strain BCI 107.

In certain aspects, the plant growth promoting Gram-negative bacteria belong to the genus Klebsiella. In one aspect, the plant growth promoting Gram-negative bacteria belong to one or more of the following species: Klebsiella oxytoca, Klebsiella pneumoniae, and Klebsiella variicola. In another aspect, the plant growth promoting Gram-negative bacteria are one or more of: Klebsiella variicola NCMA Accession No. 201708001, Klebsiella variicola NCMA Accession No. 201712001, Klebsiella variicola NCMA Accession No. 201712002, and Klebsiella oxytoca M5A1.

In certain aspects, the plant growth promoting Gram-negative bacteria belong to the genus Rahnella. In one aspect, the plant growth promoting Gram-negative bacteria belong to the following species: Rahnella aquatilis. In another aspect, the plant growth promoting Gram-negative bacteria are one or more of: Rahnella aquatilis strain CI019, Rahnella aquatilis Accession No. PTA-122293, and Rahnella aquatilis strain H145.

In certain aspects, the plant growth promoting Gram-negative bacteria belong to the genus Rhizobium. In one aspect, the plant growth promoting Gram-negative bacteria belong to the following species: Rhizobium etli, Rhizobium leguminosarum, Rhizobium phaseoli, Rhizobium tropici, Rhizobium fredii, and Rhizobium meliloti. In another aspect, the plant growth promoting Gram-negative bacteria are Rhizobium leguminosarum SO12A-2 (IDAC 080305-01).

In certain aspects, the plant growth promoting Gram-negative bacteria belong to the genus Azotobacter. In one aspect, the plant growth promoting Gram-negative bacteria belong to one or more of the following species: Azotobacter vinelandii and Azotobacter chroococcum.

In certain aspects, the plant growth promoting Gram-negative bacteria belong to the genus Massilia. In one aspect, the plant growth promoting Gram-negative bacteria belong to one or more of the following species: Massilia timonae, Massilia dura, Massilia Massilia plicata, Massilia lutea, Massilia aerilata, Massilia alkalitolerans, Massilia aurea, Massilia arvi, Massilia brevitalea, Massilia cf. timonae, Massilia consociate, Massilia eurypsychrophila, Massilia haematophila, Massilia jejuensis, Massilia kyonggiensis, Massilia lurida, Massilia niabensis, Massilia niastensis, Massilia norwichensis, Massilia oculi, Massilia putida, Massilia suwonensis, Massilia tieshanensis, Massilia umbonate, Massilia varians, and Massilia yuzhufengensis.

In certain aspects, the plant growth promoting Gram-negative bacteria belong to the genus Gluconacetobacter. In one aspect, the plant growth promoting Gram-negative bacteria belong to one or more of the following species: Gluconacetobacter aggeris, Gluconacetobacter asukensis, Gluconacetobacter azotocaptans, Gluconacetobacter diazotrophicus, Gluconacetobacter entanii, Gluconacetobacter europaeus, Gluconacetobacter hansenii, Gluconacetobacter intermedius, Gluconacetobacter johannae, Gluconacetobacter kakiaceti, Gluconacetobacter kombuchae, Gluconacetobacter liquefaciens, Gluconacetobacter maltaceti, Gluconacetobacter medellinensis, Gluconacetobacter nataicola, Gluconacetobacter oboediens, Gluconacetobacter rhaeticus, Gluconacetobacter sacchari, Gluconacetobacter saccharivorans, Gluconacetobacter sucrofermentans, Gluconacetobacter swingsii, Gluconacetobacter takamatsuzukensis, Gluconacetobacter tumulicola, Gluconacetobacter tumulisoli, and Gluconacetobacter xylinus.

In certain aspects, the plant growth promoting Gram-negative bacteria belong to the genus Acetobacter. In one aspect, the plant growth promoting Gram-negative bacteria belong to one or more of the following species: Acetobacter nitrogenifigens, Acetobacter sacchari, and Acetobacter peroxydans.

In certain aspects, the plant growth promoting Gram-negative bacteria belong to the genus Asaia. In one aspect, the plant growth promoting Gram-negative bacteria belong to one or more of the following species: Asaia siamensis, Asaia krungthepensis, Asaia lannaensis, Asaia platycodi, Asaia prunellae, and Asaia astilbes.

In certain aspects, the plant growth promoting Gram-negative bacteria belong to the genus Swaminathania. In one aspect, the plant growth promoting Gram-negative bacteria belong to one or more of the following species: S. salitorerans.

In certain aspects, the plant growth promoting Gram-negative bacteria belong to the genus Nguyenibacter vanlangensis. In one aspect, the plant growth promoting Gram-negative bacteria belong to one or more of the following species: Nguyenibacter vanlangensis.

In certain aspects, the plant growth promoting Gram-negative bacteria belong to the genus Komagataeibacter. In one aspect, the plant growth promoting Gram-negative bacteria belong to one or more of the following species: Komagataeibacter hansenii, Komagataeibacter kakiaceti, Komagataeibacter swingsii, Komagataeibacter intermedius

In certain aspects, the plant growth promoting Gram-negative bacteria belong to the genus Herbaspirillum. In one aspect, the plant growth promoting Gram-negative bacteria belong to one or more of the following species: H. aquaticum, H. autotrophicum, H. chlorophenolicum, H. frisingense, H. hiltneri, H. huttiense, H. lusitanum, H. massiliense, H. rhizosphaerae, H. rubrisubalbicans, and H. seropedicae.

In certain aspects, the plant growth promoting Gram-negative bacteria belong to the genus Lysobacter. In one aspect, the plant growth promoting Gram-negative bacteria belong to one or more of the following species: Lysobacter aestuarii, Lysobacter agri, Lysobacter antibioticus, Lysobacter arseniciresistens, Lysobacter brunescens, Lysobacter burgurensis, Lysobacter capsica, Lysobacter caeni, Lysobacter capsici, Lysobacter cavernae, Lysobacter concretionis, Lysobacter daejeonensis, Lysobacter defluvii, Lysobacter dokdonensis, Lysobacter enzymogenes, Lysobacter erysipheiresistens, Lysobacter firmicutimachus, Lysobacter fragariae, Lysobacter ginsengisoli, Lysobacter gummosus, Lysobacter hankyongensis, Lysobacter humi, Lysobacter koreensis, Lysobacter korlensis, Lysobacter lycopersici, Lysobacter maris, Lysobacter mobilis, Lysobacter niabensis, Lysobacter niastensis, Lysobacter novalis, Lysobacter olei, Lysobacter oligotrophicus, Lysobacter oryzae, Lysobacter panacisoli, Lysobacter panaciterrae, Lysobacter rhizophilus, Lysobacter rhizosphaerae, Lysobacter ruishenii, Lysobacter sediminicola, Lysobacter silvestris, Lysobacter solanacearum, Lysobacter soli, Lysobacter spongiicola, Lysobacter terrae, Lysobacter terricola, Lysobacter thermophilus, Lysobacter tolerans, Lysobacter ximonensis, Lysobacter xinjiangensis, and Lysobacter yangpyeongensis.

In certain aspects, the plant growth promoting Gram-negative bacteria belong to the genus Pantoea. In one aspect, the plant growth promoting Gram-negative bacteria belong to one or more of the following species: Pantoea alhagi, Pantoea agglomerans, Pantoea Pantoea ananatis, Pantoea anthophila, Pantoea deleyi, Pantoea dispersa, Pantoea eucalypti, Pantoea stewartia, and Pantoea intestinalis.

In certain aspects, the plant growth promoting Gram-negative bacteria belong to the genus Janthinobacterium. In one aspect, the plant growth promoting Gram-negative bacteria belong to one or more of the following species: Janthinobacterium agaricidamnosum, Janthinobacterium aquaticum, Janthinobacterium lividum, Janthinobacterium psychrotolerans, Janthinobacterium rivuli, Janthinobacterium svalbardensis, and Janthinobacterium violaceinigrum.

In certain aspects, the plant growth promoting Gram-negative bacteria belong to the genus Duganella. In one aspect, the plant growth promoting Gram-negative bacteria belong to one or more of the following species: Duganella ginsengisoli, Duganella phyllosphaerae, Duganella radices, Duganella sacchari, and Duganella zoogloeoides.

In certain aspects, the plant growth promoting Gram-negative bacteria belong to the genus Methylobacterium. In one aspect, the plant growth promoting Gram-negative bacteria belong to one or more of the following species: M. adhaesivum, M. aerolatum, M. aminovorans, M. aquaticum, M. brachiatum, M. brachythecii, M. bullatum, M. cerastii, M. dankookense, M. extorquens, M. frigidaeris, M. fujisawaense, M. gnaphalii, M. goesingense, M. gossipiicola, M. gregans, M. haplocladii, M. hispanicum, M. iners, M. isbiliense, M. jeotgali, M. komagatae, M. longum, M. marchantiae, M. mesophilicum, M. nodulans, M. organophilum, M. oryzae, M. oxalidis, M. persicinum, M. phyllosphaerae, M. phyllostachyos, M. platani, M. podarium, M. populi, M. pseudosasae, M. pseudosasicola, M. radiotolerans, M. rhodesianum, M. rhodinum, M. salsuginis, M. soli, M. suomiense, M. tardum, M. tarhaniae, M. thiocyanatum, M. thuringiense, M. trifolii, M. variabile, and M. zatmanii.

In certain aspects, the plant growth promoting Gram-negative bacteria belong to the genus Flavobacterium. In one aspect, the plant growth promoting Gram-negative bacteria belong to one or more of the following species: F. acidificum, F. aciduliphilum, F. acidurans, F. ahnfeltiae, F. algicola, F. anatoliense, F. anhuiense, F. antarcticum, F. aquaticum, F. akiainvivens, F. aquatile, F. aquicola, F. aquidurense, F. araucananum, F. arcticum, F. arsenatis, F. arsenitoxidans, F. aureus, F. banpakuense, F. beibuense, F. branchiarum, F. branchiicola, F. branchiophilum, F. breve, F. brevivitae, F. buctense, F. caeni, F. cauense, F. ceti, F. cheniae, F. cheonanense, F. cheonhonense, F. chilense, F. chungangense, F. chungbukense, F. chungnamense, F. collinsense, F. collinsii, F. columnare, F. compostarboris, F. crassostreae, F. croceum, F. cucumis, F. cutihirudinis, F. daejeonense, F. daemonensis, F. dankookense, F. defluvii, F. degerlache, F. denitrificans, F. devorans, F. dispersum, F. dongtanense, F. eburneum, F. endophyticum, F. enshiense, F. faecale, F. ferrugineum, F. filum, F. flaviflagrans, F. flevense, F. jluvil, F. fontis, F. frigidarium, F. frigidimaris, F. frigoris, F. fryxellicola, F. fulvum, F. gelidilacus, F. gillisiae, F. ginsengisoli, F. ginsenosidimutans, F. glaciei, F. glycines, F. granuli, F. halmophilum, F. haoranii, F. hauense, F. hercynium, F. hibernum, F. humicola, F. hydatis, F. indicum, F. inkyongense, F. jejuense, F. johnsoniae, F. jumunjinense, F. koreense, F. kyungheense, F. lacunae, F. lacus, F. limicola, F. limnosediminis, F. lindanitolerans, F. longum, F. luticocti, F. lutivivi, F. macrobrachii, F. maotaiense, F. marinum, F. maris, F. micromati, F. mizutaii, F. myungsuense, F. multivorum, F. nitratireducens, F. nitrogenifigens, F. noncentrifugens, F. notoginsengisoli, F. oceanosedimentum, F. omnivorum, F. oncorhynchi, F. okeanokoites, F. orientale, F. oryzae, F. palustre, F. paronense, F. pectinovorum, F. pedocola, F. phragmitis, F. piscis, F. plurextorum, F. ponti, F. procerum, F. psychrolimnae, F. psychrophilum, F. qiangtangense, F. rakeshii, F. reichenbachii, F. resisters, F. rivuli, F. saccharophilum, F. saliperosum, F. sasangense, F. segetis, F. salegens, F. seoulense, F. sinopsychrotolerans, F. soli, F. spartansii, F. squillarum, F. suaedae, F. subsaxonicum, F. succinans, F. suncheonense, F. suzhouense, F. swingsii, F. tegetincola, F. terrae, F. terrigena, F. temphilum, F. thermophilum, F. tiangeerense, F. tilapiae, F. tistrianum, F. tructae, F. tyrosinilyticum, F. ummariense, F. urocaniciphilum, F. urumqiense, F. verecundum, F. vireti, F. weaverense, F. xanthum, F. xinjiangense, F. xueshanense, F. yanchengense, and F. yonginense.

In certain aspects, the plant growth promoting Gram-negative bacteria belong to the genus Serratia. In one aspect, the plant growth promoting Gram-negative bacteria belong to one or more of the following species: S. aquatilis, S. entomophila, S. ficaria, S. fonticola, S. glossinae, S. grimesii, S. liquefaciens, S. marcescens, S. myotis, S. nematodiphila, S. odorifera, S. plymuthica, S. proteamaculans, S. quinivorans, S. rubidaea, S. symbiotica, S. ureilytica, and S. vespertilionis.

In certain aspects, the plant growth promoting Gram-negative bacteria belong to the genus Variovorax. In one aspect, the plant growth promoting Gram-negative bacteria belong to one or more of the following species: Variovorax boronicumulans, Variovorax defluvii, Variovorax dokdonensis, Variovorax ginsengisoli, Variovorax gossypii, Variovorax guangxiensis, Variovorax humicola, Variovorax paradoxus, and Variovorax soli. In another aspect, the plant growth promoting Gram-negative bacteria belongs to the species of Variovorax guangxiensis.

In certain aspects, the plant growth promoting Gram-negative bacteria belong to the genus Azospirillum. In one aspect, the plant growth promoting Gram-negative bacteria belong to one or more of the following species: Azospirillum brasilense, Azospirillum lipoferum, Azospirillum halopraeferans, and Azospirillum amazonense. In another aspect, the plant growth promoting Gram-negative bacteria are Azospirillum brasilense INTA Az-39.

In certain aspects, the plant growth promoting Gram-negative bacteria belong to the genus Enterobacter. In one aspect, the plant growth promoting Gram-negative bacteria belong to the following species: Enterobacter cloacae. In another aspect, the plant growth promoting Gram-negative bacteria are one or more of: Enterobacter cloacae strain FERM BP 1529, Enterobacter cloacae strain CAP12 (NRRL No. B-50822), and Enterobacter sp. 638.

In certain aspects, the plant growth promoting Gram-negative bacteria belong to the genus Burkholderia. In one aspect, the plant growth promoting Gram-negative bacteria belong to one or more of the following species: Burkholderia gladioli, Burkholderia oxyphila, Burkholderia sacchari, Burkholderia ferrariae, Burkholderia silvatlantica, Burkholderia heleia, Burkholderia nodosa, Burkholderia bannensis, Burkholderia tropica, Burkholderia unamae, Burkholderia kururiensis, Burkholderia diazotrophica, Burkholderia tuberum, Burkholderia acidipaludis, Burkholderia caribensis, Burkholderia hospita, Burkholderia terrae, Burkholderia phymatum, Burkholderia sabiae, Burkholderia sartisoli, Burkholderia phenazinium, Burkholderia sediminicola, Burkholderia phytofirmans, Burkholderia ginsengisoli, Burkholderia fungorum, Burkholderia megapolitana, Burkholderia bryophila, Burkholderia terricola, Burkholderia graminis, Burkholderia phenoliruptrix, Burkholderia xenovocans, Burkholderia mimosarum, Burkholderia endofungorum, Burkholderia rhizoxinica, Burkholderia soli, Burkholderia caryophlii, Burkholderia unamae, and Burkholderia caledonica. In another aspect, the plant growth promoting Gram-negative bacteria are one or more of: Burkholderia strain Q208 and Burkholderia-like species strain SOS1.

In certain aspects, the plant growth promoting Gram-negative bacteria belong to the genus Sinorhizobium. In one aspect, the plant growth promoting Gram-negative bacteria belong to one or more of the following species: Sinorhizobium fredii, Sinorhizobium medicae and Sinorhizobium meliloti. In another aspect, the plant growth promoting Gram-negative bacteria are one or more of: Sinorhizobium meliloti Rm1021, Sinorhizobium (Ensifer) meliloti strain RBD1, Sinorhizobium medicae strain NRRL Accession No. X78, Sinorhizobium meliloti strain NRRL Accession No. X79, Sinorhizobium fredii CCBAU114, and Sinorhizobium fredii USDA 205.

In certain aspects, the plant growth promoting Gram-negative bacteria belong to the genus Bradyrhizobium. In one aspect, the plant growth promoting Gram-negative bacteria belong to one or more of the following species: Bradyrhizobium diazoefficiens, Bradyrhizobium japonicum, Bradyrhizobium elkanii, Bradyrhizobium canariense, Bradyrhizobium denitrificans Bradyrhizobium iriomotense, Bradyrhizobium jicamae, Bradyrhizobium liaoningense, Bradyrhizobium pachyrhizi, and Bradyrhizobium yuanmingense. In another aspect, the plant growth promoting Gram-negative bacteria are one or more of Bradyrhizobium elkanii SEMIA 501, Bradyrhizobium elkanii SEMIA 587, Bradyrhizobium elkanii SEMIA 5019, Bradyrhizobium japonicum NRRL B-50586 (also deposited as NRRL B-59565), Bradyrhizobium japonicum NRRL B-50587 (also deposited as NRRL B-59566), Bradyrhizobium japonicum NRRL B-50588 (also deposited as NRRL B-59567), Bradyrhizobium japonicum NRRL B-50589 (also deposited as NRRL B-59568), Bradyrhizobium japonicum NRRL B-50590 (also deposited as NRRL B-59569), Bradyrhizobium japonicum NRRL B-50591 (also deposited as NRRL B-59570), Bradyrhizobium japonicum NRRL B-50592 (also deposited as NRRL B-59571), Bradyrhizobium japonicum NRRL B-50593 (also deposited as NRRL B-59572), Bradyrhizobium japonicum NRRL B-50594 (also deposited as NRRL B-50493), Bradyrhizobium japonicum NRRL B-50608, Bradyrhizobium japonicum NRRL B-50609, Bradyrhizobium japonicum NRRL B-50610, Bradyrhizobium japonicum NRRL B-50611, Bradyrhizobium japonicum NRRL B-50612, Bradyrhizobium japonicum NRRL B-50726, Bradyrhizobium japonicum NRRL B-50727, Bradyrhizobium japonicum NRRL B-50728, Bradyrhizobium japonicum NRRL B-50729, Bradyrhizobium japonicum NRRL B-50730, Bradyrhizobium japonicum SEMIA 566, Bradyrhizobium japonicum SEMIA 5079, Bradyrhizobium japonicum SEMIA 5080, Bradyrhizobium japonicum USDA 6, Bradyrhizobium japonicum USDA 110, Bradyrhizobium japonicum USDA 122, Bradyrhizobium japonicum USDA 123, Bradyrhizobium japonicum USDA 127, Bradyrhizobium japonicum USDA 129 and Bradyrhizobium japonicum USDA 532C.

### Methods of Culturing, Processing, and Formulating Microalgae

In certain aspects, the microalgae strains disclosed herein are cultured heterotrophically, mixotrophically, and/or phototrophically. In a preferred embodiment, the microalgae strains disclosed herein are cultured heterotrophically. As used herein, “heterotrophic” culturing conditions comprises supplying a culture of microorganisms with the at least one organic carbon source in the absence of a supply of light and/or carbon dioxide.

The microalgae strains can be cultured on sources of organic carbon or combinations of organic carbon sources, such as: acetate, acetic acid, ammonium linoleate, arabinose, arginine, aspartic acid, butyric acid, cellulose, citric acid, ethanol, fructose, fatty acids, galactose, glucose, glycerol, glycine, lactic acid, lactose, maleic acid, maltose, mannose, methanol, molasses, peptone, plant based hydrolysate, proline, propionic acid, ribose, saccharose, partial or complete hydrolysates of starch, sucrose, tartaric, TCA-cycle organic acids, thin stillage, urea, industrial waste solutions, yeast extract, or combinations thereof.

In some embodiments, the microalgae strains are cultured on a nitrogen source comprising monosodium glutamate (MSG), ammonia, ammonium (e.g., ammonium hydroxide, ammonium phosphate, ammonium acetate), urea, nitrates, glycine or a combination thereof. The methods of culturing the disclosed microalgae strains include methods of mixing, organic carbon supply, nitrogen supply, lighting, culture media, nutrient stocks, culturing vessels, and optimization of the culture parameters such as but not limited to temperature, pH, dissolved oxygen, and dissolved carbon dioxide. The Chlorella culture can be harvested from the culturing vessel and/or concentrated by means known in the art, such as but not limited to, settling, centrifugation, filtration, and electro-dewatering before concentration and/or drying.

In some embodiments and Examples below, a microalgae composition may be referred to as PHYCOTERRA® or PHYCOTERRA® ST. The PHYCOTERRA® or PHYCOTERRA® ST Chlorella microalgae composition is a microalgae composition comprising Chlorella. The PHYCOTERRA® product contains whole cell Chlorella biomass while the PHYCOTERRA® ST contains lysed cell Chlorella biomass. The PHYCOTERRA® Chlorella microalgae composition treatments were prepared by growing the Chlorella in non-axenic acetic acid supplied mixotrophic conditions, increasing the concentration of Chlorella using a centrifuge, pasteurizing the concentrated Chlorella at between 65° C.-75° C. for between 90-150 minutes, adding potassium sorbate and phosphoric acid to stabilize the pH of the Chlorella, and then adjusting the whole biomass treatment to the desired concentration. The PHYCOTERRA® Chlorella microalgae composition may comprise approximately 10% w/w of Chlorella microalgae cells. Furthermore, the PHYCOTERRA® Chlorella microalgae composition may comprise between approximately 0.3% potassium sorbate and between approximately 0.5%-1.5% phosphoric acid to stabilize the pH of the Chlorella to between 3.0-4.0 and 88.2%-89.2% water. It should be clearly understood, however, that other variations of the PHYCOTERRA® Chlorella microalgae composition, including variations in the microalgae strains, variations in the stabilizers, and/or variations in the % composition of each component may be used and may achieve similar results.

A composition comprising microalgae can be stabilized by heating and cooling in a pasteurization process. In certain aspects, the active ingredients of the microalgae based compositions maintain effectiveness in enhancing at least one characteristic of a plant after being subjected to the heating and cooling of a pasteurization process. In other embodiments, compositions with whole cells or processed cells (e.g., dried, lysed, extracted) of microalgae cells may not need to be stabilized by pasteurization. For example, microalgae cells that have been processed, such as by drying, lysing, and extraction, or extracts can include such low levels of bacteria that a composition can remain stable without being subjected to the heating and cooling of a pasteurization process.

In some embodiments, the composition is lysed. Lysing is a technique where the cell membrane of a cell is ruptured, which releases lysate, the fluid contents of lysed cells, from the cells. As an example, the lysing process can comprise anything suitable that ruptures a cell membrane. For example, a bead mill may be used for lysing, where feedstock biomass solids can be dispersed and wetted (e.g., placed into a liquid phase). In this example the bead mill can utilize ceramic, glass, or metal beats (e.g., of a suitable size for the desired result) disposed in a chamber, such as a rotating cylinder, to collide with and mechanically macerate the solid biomass in the mill, which can help rupture the cell walls (e.g., the hydrogen bonds that hold together a cell membrane). Accordingly, in this example, the whole biomass may be lysed with water at cooler temperatures, with the resulting lysate comprising lipids in the form of an oil, biomass cell contents and unbroken biomass solid (e.g., non-target portion of biomass), and water.

In another aspect, the biomass is lysed using a shear mill. A shear mill utilizes a rotating impeller or high-speed rotor to create flow and shear of its contents. This causes the solid particles, such as biomass solid, to rupture due to shear stress.

In another aspect, the biomass is lysed using a pulsed electron field (PEF), high pressure homogenization, enzymes, and/or a chemical means (e.g., with a solvent).

In some embodiments, the composition can be heated to a temperature in the range of 50-130° C. In some embodiments, the composition can be heated to a temperature in the range of 55-65° C. In some embodiments, the composition can be heated to a temperature in the range of 58-62° C. In some embodiments, the composition can be heated to a temperature in the range of 50-60° C. In some embodiments, the composition can be heated to a temperature in the range of 60-90° C. In some embodiments, the composition can be heated to a temperature in the range of 70-80° C. In some embodiments, the composition can be heated to a temperature in the range of 100-150° C. In some embodiments, the composition can be heated to a temperature in the range of 120-130° C.

After the step of heating or subjecting the composition to high temperatures is complete, the compositions can be cooled at any rate to a temperature that is safe to work with. In one non-limiting embodiment, the composition can be cooled to a temperature in the range of 35-45° C. In some embodiments, the composition can be cooled to a temperature in the range of 36-44° C. In some embodiments, the composition can be cooled to a temperature in the range of 37-43° C. In some embodiments, the composition can be cooled to a temperature in the range of 38-42° C. In some embodiments, the composition can be cooled to a temperature in the range of 39-41° C. In further embodiments, the pasteurization process can be part of a continuous production process that also involves packaging, and thus the composition can be packaged (e.g., bottled) directly after the heating or high temperature stage without a cooling step.

In some embodiments, the composition can include 2.5-30% solids by weight of microalgae cells (i.e., 2.5-30 g of microalgae cells/100 mL of the composition). In some embodiments, the composition can include 2.5-5% solids by weight of microalgae cells (i.e., 2.5-5 g of microalgae cells/100 mL of the composition). In some embodiments, the composition can include 5-20% solids by weight of microalgae cells. In some embodiments, the composition can include 5-15% solids by weight of microalgae cells. In some embodiments, the composition can include 5-10% solids by weight of microalgae cells. In some embodiments, the composition can include 10-20% solids by weight of microalgae cells. In some embodiments, the composition can include 10-20% solids by weight of microalgae cells. In some embodiments, the composition can include 20-30% solids by weight of microalgae cells. In some embodiments, further dilution of the microalgae cells percent solids by weight can occur before application for low concentration applications of the composition.

In some embodiments, the composition can include less than 1% by weight of microalgae biomass or extracts (i.e., less than 1 g of microalgae derived product/100 mL of the composition). In some embodiments, the composition can include less than 0.9% by weight of microalgae biomass or extracts. In some embodiments, the composition can include less than 0.8% by weight of microalgae biomass or extracts. In some embodiments, the composition can include less than 0.7% by weight of microalgae biomass or extracts. In some embodiments, the composition can include less than 0.6% by weight of microalgae biomass or extracts. In some embodiments, the composition can include less than 0.5% by weight of microalgae biomass or extracts. In some embodiments, the composition can include less than 0.4% by weight of microalgae biomass or extracts. In some embodiments, the composition can include less than 0.3% by weight of microalgae biomass or extracts. In some embodiments, the composition can include less than 0.2% by weight of microalgae biomass or extracts. In some embodiments, the composition can include less than 0.1% by weight of microalgae biomass or extracts. In some embodiments, the composition can include at least 0.0001% by weight of microalgae biomass or extracts. In some embodiments, the composition can include at least 0.001% by weight of microalgae biomass or extracts. In some embodiments, the composition can include at least 0.01% by weight of microalgae biomass or extracts. In some embodiments, the composition can include at least 0.1% by weight of microalgae biomass or extracts. In some embodiments, the composition can include 0.0001-1% by weight of microalgae biomass or extracts. In some embodiments, the composition can include 0.0001-0.001% by weight of microalgae biomass or extracts. In some embodiments, the composition can include 0.001-0.01% by weight of microalgae biomass or extracts. In some embodiments, the composition can include 0.01-0.1% by weight of microalgae biomass or extracts. In some embodiments, the composition can include 0.1-1% by weight of microalgae biomass or extracts.

In some embodiments, an application concentration of 0.1% of microalgae biomass or extract equates to 0.04 g of microalgae biomass or extract in 40 mL of a composition. While the desired application concentration to a plant can be 0.1% of microalgae biomass or extract, the composition can be packaged as a 10% concentration (0.4 mL in 40 mL of a composition). Thus, a desired application concentration of 0.1% would require 6,000 mL of the 10% microalgae biomass or extract in the 100 gallons of water applied to the assumption of 15,000 plants in an acre, which is equivalent to an application rate of about 1.585 gallons per acre. In some embodiments, a desired application concentration of 0.01% of microalgae biomass or extract using a 10% concentration composition equates to an application rate of about 0.159 gallons per acre. In some embodiments, a desired application concentration of 0.001% of microalgae biomass or extract using a 10% concentration composition equates to an application rate of about 0.016 gallons per acre. In some embodiments, a desired application concentration of 0.0001% of microalgae biomass or extract using a 10% concentration composition equates to an application rate of about 0.002 gallons per acre.

In another non-limiting embodiment, correlating the application of the microalgae biomass or extract on a per plant basis using the assumption of 15,000 plants per acre, the composition application rate of 1 gallon per acre is equal to about 0.25 mL per plant=0.025 g per plant=25 mg of microalgae biomass or extract per plant. The water requirement assumption of 100 gallons per acre is equal to about 35 mL of water per plant. Therefore, 0.025 g of microalgae biomass or extract in 35 mL of water is equal to about 0.071 g of microalgae biomass or extract per 100 mL of composition equates to about a 0.07% application concentration. In some embodiments, the microalgae biomass or extract-based composition can be applied at a rate in a range as low as about 0.001-10 gallons per acre, or as high as up to 150 gallons per acre.

In some embodiments, the applications are performed using a 10% solids solution by weight microalgae composition. For greenhouse trials, the rates vary and essentially refer to how much volume of the 10% solids solution are added in a given volume of water (e.g. 2.5% v/v-5.0% v/v).

The present invention involves the use of a microalgae composition. Microalgae compositions, methods of preparing microalgae compositions, and methods of applying the microalgae compositions to plants are disclosed in WO 2017/218896 A1 (Shinde et al.) entitled “Microalgae-Based Composition, and Methods of its Preparation and Application to Plants,” which is incorporated herein in full by reference. In one or more embodiments, the microalgae composition may comprise approximately 10%-10.5% w/w of Chlorella microalgae cells. In one or more embodiments, the microalgae composition may also comprise one of more stabilizers, such as potassium sorbate, phosphoric acid, ascorbic acid, sodium benzoate, citric acid, or the like, or any combination thereof. For example, in one or more embodiments, the microalgae composition may comprise approximately 0.3% w/w of potassium sorbate or another similar compound to stabilize its pH and may further comprise approximately 0.5-1.5% w/w phosphoric acid or another similar compound to prevent the growth of contaminants. As a further example, in one or more embodiments where it is desired to use an OMRI (Organic Materials Review Institute) certified organic composition, the microalgae composition may comprise 1.0-2.0% w/w citric acid to stabilize its pH and may not contain potassium sorbate or phosphoric acid. In one or more embodiments, the pH of the microalgae composition may be stabilized to between 3.0-4.0.

In some embodiments, the composition is a liquid and substantially includes of water. In some embodiments, the composition can include 70-99% water. In some embodiments, the composition can include 85-95% water. In some embodiments, the composition can include 70-75% water. In some embodiments, the composition can include 75-80% water. In some embodiments, the composition can include 80-85% water. In some embodiments, the composition can include 85-90% water. In some embodiments, the composition can include 90-95% water. In some embodiments, the composition can include 95-99% water. The liquid nature and high-water content of the composition facilitates administration of the composition in a variety of manners, such as but not limit to: flowing through an irrigation system, flowing through an above ground drip irrigation system, flowing through a buried drip irrigation system, flowing through a central pivot irrigation system, sprayers, sprinklers, and water cans.

In some embodiments, the composition can be used immediately after formulation, or can be stored in containers for later use. In some embodiments, the composition can be stored out of direct sunlight. In some embodiments, the composition can be refrigerated. In some embodiments, the composition can be stored at 1-10° C. In some embodiments, the composition can be stored at 1-3° C. In some embodiments, the composition can be stored at 3-50° C. In some embodiments, the composition can be stored at 5-8° C. In some embodiments, the composition can be stored at 8-10° C.

### Methods of Application and Application Rates for Plants

In some embodiments, administration of the composition to soil, a seed or plant can be in an amount effective to produce an enhanced characteristic in plants compared to a substantially identical population of untreated seeds or plants. Such enhanced characteristics can include accelerated seed germination, accelerated seedling emergence, improved seedling emergence, improved leaf formation, accelerated leaf formation, improved plant maturation, accelerated plant maturation, increased plant yield, increased plant growth, increased plant quality, increased plant health, increased fruit yield, increased fruit sweetness, increased fruit growth, and increased fruit quality. Non-limiting examples of such enhanced characteristics can include accelerated achievement of the hypocotyl stage, accelerated protrusion of a stem from the soil, accelerated achievement of the cotyledon stage, accelerated leaf formation, increased marketable plant weight, increased marketable plant yield, increased marketable fruit weight, increased production plant weight, increased production fruit weight, increased utilization (indicator of efficiency in the agricultural process based on ratio of marketable fruit to unmarketable fruit), increased chlorophyll content (indicator of plant health), increased plant weight (indicator of plant health), increased root weight (indicator of plant health), increased shoot weight (indicator of plant health), increased plant height, increased thatch height, increased resistance to salt stress, increased plant resistance to heat stress (temperature stress), increased plant resistance to heavy metal stress, increased plant resistance to drought, increased plant resistance to disease, improved color, reduced blossom end rot, and reduced sun burn. Such enhanced characteristics can occur individually in a plant, or in combinations of multiple enhanced characteristics.

Additionally, the present invention is directed to a method of treating a plant, a plant part, such as a seed, root, rhizome, corm, bulb, or tuber, and/or a locus on which or near which the plant or the plant parts grow, such as soil, to enhance plant growth, the method comprising the step of applying to a plant, a plant part and/or a plant locus a composition comprising an isolated biologically pure culture of Parachlorella kessleri Accession No. NCMA 202103001, a mutant thereof, or a cell-free preparation or extracellular polymeric substance thereof.

The compositions disclosed herein may be applied in any desired manner, such as in the form of a seed coating, soil drench, and/or directly in-furrow and/or as a foliar spray and applied either pre-emergence, post-emergence or both. In other words, the compositions can be applied to the seed, the plant or to the soil wherein the plant is growing or wherein it is desired to grow (plant's locus of growth).

In some embodiments, the microalgae-based composition may be applied to soil, seeds, and plants in an in-furrow application. An application of the microalgae-based composition in-furrow requires a low amount of water and targets the application to a small part of the field. The application in-furrow also concentrates the application of the microalgae-based composition at a place where the seedling radicles and roots will pick up the material in the composition or make use of captured nutrients, including phytohormones.

In some embodiments, the microalgae-based composition may be applied to soil, seeds, and plants as a side dress application. One of the principals of plant nutrient applications is to concentrate the nutrients in an area close to the root zone so that the plant roots will encounter the nutrients as the plant grows. Side-dress applications use a “knife” that is inserted into the soil and delivers the nutrients around 2 inches along the row and about 2 inches or more deep. Side-dress applications are made when the plants are young and prior to flowering to support yield. Side-dress applications can only be made prior to planting in drilled crops, i.e., wheat and other grains, and alfalfa, but in row crops such as peppers, corn, tomatoes they can be made after the plants have emerged.

In some embodiments, the microalgae-based composition may be applied to soil, seeds, and plants through a drip system. Depending on the soil type, the relative concentrations of sand, silt and clay, and the root depth, the volume that is irrigated with a drip system may be about ⅓ of the total soil volume. The soil has an approximate weight of 4,000,000 lbs. per acre one foot deep. Because the roots grow where there is water, the plant nutrients in the microalgae-based composition would be delivered to the root system where the nutrients will impact most or all of the roots. Experimental testing of different application rates to develop a rate curve would aid in determining the optimum rate application of a microalgae-based composition in a drip system application.

In some embodiments, the microalgae-based composition may be applied to soil, seeds, and plants through a pivot irrigation application. The quantity and frequency of water delivered over an area by a pivot irrigation system is dependent on the soil type and crop. Applications may be 0.5 inch or more and the exact demand for water can be quantitatively measured using soil moisture gauges. For crops such as alfalfa that are drilled in (very narrow row spacing), the roots occupy the entire soil area. Penetration of the soil by the microalgae-based composition may vary with a pivot irrigation application but would be effective as long as the application can target the root system of the plants. In some embodiments, the microalgae-based composition may be applied in a broadcast application to plants with a high concentration of plants and roots, such as row crops.

In some embodiments, a composition can be administered before the seed is planted. In some embodiments, a composition can be administered at the time the seed is planted. In some embodiments, a composition can be applied by dip treatment of the roots. In some embodiments, a composition can be administered to plants that have emerged from the ground. In some embodiments, a liquid or dried composition can be applied to the soil before, during, or after the planting of a seed. In some embodiments a liquid or dried composition can be applied to the soil before or after a plant emerges from the soil.

In some embodiments, the volume or mass of the microalgae-based composition applied to a seed, seedling, or plant may not increase or decrease during the growth cycle of the plant (i.e., the amount of the microalgae composition applied to the plant will not change as the plant grows larger). In some embodiments, the volume or mass of the microalgae-based composition applied to a seed, seedling, or plant can increase during the growth cycle of the plant (i.e., applied on a mass or volume per plant mass basis to provide more of the microalgae composition as the plant grows larger). In some embodiments, the volume or mass of the microalgae-based composition applied to a seed, seedling, or plant can decrease during the growth cycle of the plant (i.e., applied on a mass or volume per plant mass basis to provide more of the microalgae composition as the plant grows larger).

In one non-limiting embodiment, the administration of the composition may comprise contacting the foliage of the plant with an effective amount of the composition. In some embodiments, the composition may be sprayed on the foliage by a hand sprayer, a sprayer on an agriculture implement, or a sprinkler. In some embodiments, the composition can be applied to the soil.

In certain aspects, the microalgae-based composition is applied at 0.1-150 gallons per acre, 0.1-50 gallons per acre, or 0.1-10 gallons per acre.

The rate of application of the composition at the desired concentration can be expressed as a volume per area. In some embodiments, the rate of application of the composition in a foliar application or a soil application can comprise a rate in the range of 10-50 gallons/acre. In some embodiments, the rate of application of the composition in a foliar application can comprise a rate in the rage of 10-15 gallons/acre. In some embodiments, the rate of application of the composition in a foliar application can comprise a rate in the range of 15-20 gallons/acre. In some embodiments, the rate of application of the composition in a foliar application can comprise a rate in the range of 20-25 gallons/acre. In some embodiments, the rate of application of the composition in a foliar application can comprise a rate in the range of 25-30 gallons/acre. In some embodiments, the rate of application of the composition in a foliar application can comprise a rate in the range of 30-35 gallons/acre. In some embodiments, the rate of application of the composition in a foliar application can comprise a rate in the range of 35-40 gallons/acre. In some embodiments, the rate of application of the composition in a foliar application can comprise a rate in the range of 40-45 gallons/acre. In some embodiments, the rate of application of the composition in a foliar application can comprise a rate in the range of 45-50 gallons/acre.

In some embodiments, the rate of application of the composition in a foliar application or a soil application can include a rate in the range of 0.01-10 gallons/acre. In some embodiments, the rate of application of the composition in a soil application can include a rate in the range of 0.01-0.1 gallons/acre. In some embodiments, the rate of application of the composition in a soil application can include a rate in the range of 0.1-1.0 gallons/acre. In some embodiments, the rate of application of the composition in a soil application can include a rate in the range of 1-2 gallons/acre. In some embodiments, the rate of application of the composition in a soil application can include a rate in the range of 2-3 gallons/acre. In some embodiments, the rate of application of the composition in a soil application can include a rate in the range of 3-4 gallons/acre. In some embodiments, the rate of application of the composition in a soil application can include a rate in the range of 4-5 gallons/acre. In some embodiments, the rate of application of the composition in a soil application can include a rate in the range of 5-10 gallons/acre.

In some embodiments, the rate of application of the composition in a soil application can include a rate in the range of 2-20 liters/acre. In some embodiments, the rate of application of the composition in a soil application can include a rate in the range of 3.7-15 liters/acre. In some embodiments, the rate of application of the composition in a soil application can include a rate in the range of 2-5 liters/acre. In some embodiments, the rate of application of the composition in a soil application can include a rate in the range of 5-10 liters/acre. In some embodiments, the rate of application of the composition in a soil application can include a rate in the range of 10-15 liters/acre. In some embodiments, the rate of application of the composition in a soil application can include a rate in the range of 15-20 liters/acre.

In some embodiments, the v/v ratio of the composition can be between 0.001%-50%. The v/v ratio can be between 0.01-25%. The v/v ratio of the composition can be between 0.03-10%.

In another non-limiting embodiment, the administration of the composition can include contacting the soil in the immediate vicinity of the planted seed with an effective amount of the composition. In some embodiments, the composition can be supplied to the soil by injection into a low volume irrigation system, such as but not limited to a drip irrigation system supplying water beneath the soil through perforated conduits or at the soil level by fluid conduits hanging above the ground or protruding from the ground. In some embodiments, the composition can be supplied to the soil by a soil drench method wherein the composition is poured on the soil.

The composition can be diluted to a lower concentration for an effective amount in a soil application by mixing a volume of the composition in a volume of water. The percent solids of microalgae sourced components resulting in the diluted composition can be calculated by the multiplying the original concentration in the composition by the ratio of the volume of the composition to the volume of water. Alternatively, the grams of microalgae sourced components in the diluted composition can be calculated by the multiplying the original grams of microalgae sourced components per 100 mL by the ratio of the volume of the composition to the volume of water.

Plants Benefitting from Application of the Compositions

Many plants can benefit from the application of compositions that provide a bio-stimulatory effect. Non-limiting examples of plant families that can benefit from such compositions include plants from the following: Solanaceae, Fabaceae (Leguminosae), Poaceae, Roasaceae, Vitaceae, Brassicaeae (Cruciferae), Caricaceae, Malvaceae, Sapindaceae, Anacardiaceae, Rutaceae, Moraceae, Convolvulaceae, Lamiaceae, Verbenaceae, Pedaliaceae, Asteraceae (Compositae), Apiaceae (Umbelliferae), Araliaceae, Oleaceae, Ericaceae, Actinidaceae, Cactaceae, Chenopodiaceae, Polygonaceae, Theaceae, Lecythidaceae, Rubiaceae, Papveraceae, Illiciaceae Grossulariaceae, Myrtaceae, Juglandaceae, Bertulaceae, Cucurbitaceae, Asparagaceae (Liliaceae), Alliaceae (Liliceae), Bromeliaceae, Zingieraceae, Muscaceae, Areaceae, Dioscoreaceae, Myristicaceae, Annonaceae, Euphorbiaceae, Lauraceae, Piperaceae, Proteaceae, and Cannabaceae.

The Solanaceae plant family includes a large number of agricultural crops, medicinal plants, spices, and ornamentals in its over 2,500 species. Taxonomically classified in the Plantae kingdom, Tracheobionta (subkingdom), Spermatophyta (superdivision), Magnoliophyta (division), Manoliopsida (class), Asteridae (subclass), and Solanales (order), the Solanaceae family includes, but is not limited to, potatoes, tomatoes, eggplants, various peppers, tobacco, and petunias. Plants in the Solanaceae can be found on all the continents, excluding Antarctica, and thus have a widespread importance in agriculture across the globe.

The Rosaceae plant family includes flowering plants, herbs, shrubs, and trees. Taxonomically classified in the Plantae kingdom, Tracheobionta (subkingdom), Spermatophyta (superdivision), Magnoliophyta (division), Magnoliopsida (class), Rosidae (subclass), and Rosales (order), the Rosaceae family includes, but is not limited to, almond, apple, apricot, blackberry, cherry, nectarine, peach, plum, raspberry, strawberry, and quince.

The Fabaceae plant family (also known as the Leguminosae) comprises the third largest plant family with over 18,000 species, including a number of important agricultural and food plants. Taxonomically classified in the Plantae kingdom, Tracheobionta (subkingdom), Spermatophyta (superdivision), Magnoliophyta (division), Manoliopsida (class), Rosidae (subclass), and Fabales (order), the Fabaceae family includes, but is not limited to, soybeans, beans, green beans, peas, chickpeas, alfalfa, peanuts, sweet peas, carob, and liquorice. Plants in the Fabaceae family can range in size and type, including but not limited to, trees, small annual herbs, shrubs, and vines, and typically develop legumes. Plants in the Fabaceae family can be found on all the continents, excluding Antarctica, and thus have a widespread importance in agriculture across the globe. Besides food, plants in the Fabaceae family can be used to produce natural gums, dyes, and ornamentals.

The Poaceae plant family supplies food, building materials, and feedstock for fuel processing. Taxonomically classified in the Plantae kingdom, Tracheobionta (subkingdom), Spermatophyta (superdivision), Magnoliophyta (division), Liliopsida (class), Commelinidae (subclass), and Cyperales (order), the Poaceae family includes, but is not limited to, flowering plants, grasses, and cereal crops such as barely, corn, lemongrass, millet, oat, rye, rice, wheat, sugarcane, and sorghum. Types of turf grass found in Arizona include, but are not limited to, hybrid Bermuda grasses (e.g., 328 tifgrn, 419 tifway, tif sport).

The Vitaceae plant family includes flowering plants and vines. Taxonomically classified in the Plantae kingdom, Tracheobionta (subkingdom), Spermatophyta (superdivision), Magnoliophyta (division), Magnoliopsida (class), Rosidae (subclass), and Rhammales (order), the Vitaceae family includes, but is not limited to, grapes.

In certain aspects, any of a variety of plants may benefit from the workings of the composition according to the invention. In one embodiment, the plant is an ornamental plant, which includes flowering and non-flowering plants. In another embodiment, the plant is a consumable plant, which includes cereals, crops, fruit trees, herbs, medicinal plants and vegetables. In another embodiment, the plant is a member of the Alliaceae, Apiaceae, Asparagaceae, Asphodelaceae, Asteraceae, Araucariaceae, Begoniaceae, Brassicaceae, Bromeliaceae, Buxaceae, Chenopidiaceae, Cichorioideae, Chenopodiaceae, Coniferae, Cucurbitaceae, Fabaceae, Gentianaceae, Gramineaejridaceae, Leguminosae, Liliaceae, Malvaceae, Marantaceae, Marasmiaceae, Musaceae, Oleaceae, Orchidaceae, Paeoniaceae, Pleurotaceae, Pinaceae, Poaceae, Rosaceae, Rubiaceae, Rutaceae, Salicaceae, Solanaceae, Sterculiaceae, Taxaceae, Tuberacea, Vandeae, Vitacea or Xanthorrhoeaceae family, preferably of the Asteraceae, Begoniaceae, Brassicaceae, Chenopodiaceae, Cucurbitaceae, Gramineae, Leguminosae, Liliaceae, Malvaceae, Musaceae, Orchidaceae, Paeoniaceae, Rosaceae, Rubiaceae, Rutaceae, Salicaceae, Solanaceae, Sterculiaceae or Vandeae family, most preferably of the Begoniaceae, Brassicaceae, Orchidaceae, Paeoniaceae, Rosaceae or Solanaceae family. The plant may be a species of the genus Alchemilla, Allium, Aloe, Alstroemeria, Arabidopsis, Argyranthemum, Avena, Begonia, Brassica, Bromelia, Buxus, Calathea, Campanula, Capsicum, Cattleya, Cichorium, Citrus, Chamaecyparis, Chrysanthemum, Clematis, Cucumis, Cyclamen, Cydonia, Cymbidium, Cynodon, Dianthus, Dracaena, Eriobotrya, Euphorbia, Eustoma, Ficus, Fragaria, Fuchsia, Gaultheria, Gerbera, Glycine, Gypsophilia, Hedera, Helianthus, Hordeum, Hyacinthus, Hydrangea, Hippeastrum, Iris, Kalanchoe, Lactuca, Lathyrus, Lavendula, Lilium, Limonium, Malus, Mandevilla, Olea, Oryza, Osteospermum, Paeonia, Panicum, Pelargonium, Petunia, Phalaenopsis, Phaseolus, Pinus, Pisum, Platycodon, Prunus, Pyrus, Ranunculus, Rhododendron, Rosa, Rubus, Ruta, Secale, Skimmia, Solanum, Sorbus, Sorghum, Spathiphyllum, Trifolium, Triticum, Tulipa, Vanda, Vicia, Viola, Vitis, Zamioculcas or Zea. Preferably, the plant is a species of Arabidopsis, Begonia, Brassica, Fragaria, Paeonia, Phalaenopsis, Rosa, Solanum or Vanda.

In particular, the composition may be used to promote the growth of commercially important crops and plants, such as alfalfa, apples, bananas, begonias, bromeliads, cereals, cherries, citrus fruits, grapes, maize, melons, olives, onions, orchids, peaches, peonies, potatoes, rice, soybeans, canola, sugar beets, spinach, strawberries, tomatoes or wheat.

The composition according to the invention may also be used for improving the growth or development of seeds, tubers or bulbs. The composition may be used as such or may be mixed with substrate or nutrition medium. It may be applied to the seeds, tubers or bulbs in any convenient way, including pouring, soaking and spraying. In one embodiment, the composition according to the invention is used to coat seeds, tubers or bulbs.

The effect of the application of the composition according to the invention is improved growth, such as improved root development, improved nutrient assimilation, improved efficiency of plant metabolism or increased photosynthesis. This may be apparent from improved yield, improved leaf formation, improved color formation, improved flowering, improved fruit formation, improved taste or improved health compared to a similar plant to which the liquid composition according to the invention has not been applied.

Improvements may be determined in any suitable way generally used by the person skilled in the art, for example by counting, weighing or measuring. Improvement in any one of these areas may be at least 5%, at least 10%, at least 15%, at least 20%, at least 30%, at least 40%, at least 50%, at least 60%, at least 70%, at least 80%, at least 90%, at least 100%, at least 200%, or at least 300%, such as about 5% to 50%, about 5% to 100%, about 10% to 100%, about 20% to 50%, about 20% to 100% or about 100% to 200%.

Improved root development may be reflected in several ways, such as by more roots per plant, more roots per square area, accelerated root formation, earlier root formation, stronger roots, thicker roots, better functioning roots, more branched roots or a wider spread root network.

Improved yield may be reflected in several ways, such as by more plants per area, more branches per plant, more buds per plant, more bulbs per plant, more fruits per plant, more flowers per plant, more leaves per plant, more seedlings from seed, more seeds per plant, more shoots per plant, more spores per plant, more starch per plant, more tubers per plant, more weight per plant, higher dry matter content, more primary metabolites per plant or more secondary metabolites per plant.

Improved growth may be reflected in several ways, such as by earlier germination, accelerated germination, accelerated stem growth, a thicker stem, earlier fruit formation, accelerated fruit formation, earlier ripening of fruit or accelerated ripening of fruit.

Improved leaf formation may be reflected in several ways, such as by more leaves per plant, more leaves per cm of stem, more buds per stem, larger leaves, broader leaves, thicker leaves, stronger leaves, better functioning leaves or earlier or accelerated leaf formation.

Improved color formation may be reflected in several ways, such as by earlier color formation, accelerated color formation, more diverse color formation, deeper color formation, more intense color or more stability of color.

Improved flowering may be reflected in several ways, such as by earlier flowering, accelerated flowering, larger flowers, more flowers, more open flowers, longer lasting flowers, longer open flowers, by flowers which are more diverse in color, by flowers having a desired color or by flowers with more stability of color.

Improved fruit formation may be reflected in several ways, such as by earlier fruit formation, accelerated fruit formation, longer period of bearing fruit, earlier ripening of fruit, accelerated ripening of fruit, more fruit, heavier fruit, larger fruit or tastier fruit.

Improved taste may be reflected in several ways, such as by less acidity, more sweetness, more flavor, more complex flavor profile, higher nutrient content or more juiciness.

Improved health may be reflected in several ways, such as by being more resistant to abiotic stress, being more resistant to biotic stress, being more resistant to chemical stress, being more resistant to physical stress, being more resistant to physiological stress, being more resistant to insect pests, being more resistant to fungal pests, growing more abundantly, flowering more abundantly, keeping leaves for a longer period or being more efficient in food uptake. In the present context, biotic stress factors include fungi and insects. Abiotic stress is the result of salinity, temperature, water or light conditions which are extreme to the plant under the given circumstances.

In one embodiment, the use of the composition according to the invention leads to harvesting more plants or plant parts per area, such as more barks, berries, branches, buds, bulbs, cut branches, cut flowers, flowers, fruits, leaves, roots, seeds, shoots, spores or tubers per plant per area. The use of the liquid composition according to the invention may lead to an increase in the yield of crops. The harvest may be more abundant, and harvesting may take place after a shorter period of time, in comparison with a situation in which the composition according to the invention is not applied.

In one embodiment, application of the liquid composition according to the invention leads to more kilos of flowers, fruits, grains or vegetables, such as apples, auberges, bananas, barley, bell peppers, blackberries, blue berries, cherries, chives, courgettes, cucumber, endive, garlic, grapes, leek, lettuce, maize, melons, oats, onions, oranges, pears, peppers, potatoes, pumpkins, radish, raspberries, rice, rye, strawberries, sweet peppers, tomatoes or wheat.

In another embodiment, the application of the method according to the invention leads to more kilos of barks, berries, branches, buds, flowers, fruits, leaves, roots or seeds from culinary or medicinal herbs, such as basil, chamomile, catnip, chives, coriander, dill, eucalyptus, fennel, jasmine, lavas, lavender, mint, oregano, parsley, rosemary, sage, thyme and thus to more aroma, flavor, fragrance, oil or taste in the same period of time or in a shorter period of time, in comparison to a situation in which the composition according to the invention has not been applied.

In another embodiment, the use of the liquid composition according to the invention leads to a higher yield of antioxidants, colorants, nutrients, polysaccharides, pigments or terpenes. In one embodiment, the sugar content of the plant cells is increased.

The period of comparison with a control plant or control situation may be any period, from several hours, several days or several weeks to several months or several years. The area of comparison may be any area, such as square meters or hectares or per pot.

### Methods of Improving Soil Health

Soil health (also known as soil quality) tests have traditionally focused on soil texture, chemical concentrations such as nitrogen (N), phosphorus (P), potassium (K), and other macro or micronutrients, etc. Adding inorganic fertilizers and tilling agricultural land increased productivity in the short run, but these practices had a negative impact on soil health and productivity in the long run. Current agricultural management practices focus on conserving and improving soil by treating it as a living system. The soil microbiome and its interactions with abiotic factors of soil provide insights into improving stable soil organic matter and water conservation. The suite of soil health assays used herein put the interactions between physical, chemical, and biological factors at the forefront of soil quality analysis. These are used to directly measure impacts of microalgae products on overall soil quality.

Physical components of soil quality are tested to determine attributes beyond simple soil texture. The water holding capacity of a soil is a very important agronomic characteristic. Total water holding capacity estimates the amount of moisture held in soil after drainage and has a direct relationship to plant available water (Viji et al 2012). Total dissolved solids and total suspended solids approximate the amount of nutrients and soil lost to runoff water. Testing soil aggregation yields important information on how soil influences and is influenced by the microorganisms inside it (Dinel et al 1992), as well as how effectively air and water exchange take place within the soil. Assessment of pH and electroconductivity is essential to monitoring soil health due to the significant effects on agricultural productivity.

Tests examining the biological factors present in soil, active carbon and soil protein, are particularly important as major soil health indicators. Active carbon, as labile soil organic carbon, serves as a readily available food and energy source for the soil microbial community. It is primarily influenced by ‘new’ organic matter (originating from plants, animals, or biological products used in the field). Active carbon is highly correlated with particular organic matter (POM), which is determined with a more complex and labor-intensive chemical extraction procedure. Soil protein content is well associated with overall soil health status because of its indication of biological and chemical soil health, in particular, the quality of soil organic matter. As organically bound nitrogen, it influences the ability of the soil to store nitrogen and make it available for plant uptake through soil microbial activity. Both tests respond to soil and crop management much more rapidly than total organic matter and have been associated with soil aggregation and therefore water storage and movement.

Combining the information from all these tests creates a more complete and concise view of overall soil health and how different factors combine to create changes in the soil. Modern farming practices put a new focus on the importance of organic matter in supporting healthy and sustainable agriculture. However, measuring changes due to organic matter in soil requires a significant amount of time (often years). The assays detailed in this document provide a more up-to-the-minute look into soil health and supply valuable information on the relationship between physical, chemical, and biological properties and how these properties are altered by the addition of various microalgae products.

To achieve these improvements in soil health, an isolated biologically pure culture of Parachlorella kessleri Accession No. NCMA 202103001, a mutant thereof having all the identifying characteristics thereof, or a cell-free preparation or extracellular polymeric substance thereof, in a dried or liquid solution form are used. Microalgae can be grown in heterotrophic, mixotrophic, and phototrophic conditions. Culturing microalgae in heterotrophic conditions comprises supplying organic carbon (e.g., acetic acid, acetate, glucose) to cells in an aqueous culture medium comprising trace metals and nutrients (e.g., nitrogen, phosphorus). Culturing microalgae in mixotrophic conditions comprises supplying light and organic carbon (e.g., acetic acid, acetate, glucose) to cells in an aqueous culture medium comprising trace metals and nutrients (e.g., nitrogen, phosphorus). Culturing microalgae in phototrophic conditions comprises supplying light and inorganic carbon (e.g., carbon dioxide) to cells in an aqueous culture medium comprising trace metals and nutrients (e.g., nitrogen, phosphorus).

Improvements in soil health may be measured as at least one of: increase an amount of active carbon in the soil, increase an amount of protein in the soil, increase an amount of culturable bacteria in the soil, decrease in soil crusting, decrease in soil compaction, decrease an amount of total suspended solids lost in run-off from the soil, decrease an amount of total dissolved solids lost in run-off from the soil, increase water holding capacity of the soil, and increase soil aggregation.

In one aspect, soil crusting and/or soil compaction are decreased in treated soil. By decreasing levels of soil crusting and/or compaction, the compositions disclosed herein improve plant germination rates and emergence rates in the soil. The soil crusting and soil compaction can be measured with a penetrometer. There are many types of penetrometer designed to be used on soil. They are usually round or cone shaped. The penetrometer is dropped on the test subject or pressed against it and the depth of the resulting hole is measured or the amount of pressure (e.g., pounds per square inch) needed to penetrate the surface is reported back to the user.

It is important to draw a distinction between soil crusting and compaction. Soil compaction is caused by compressive forces on the soil such as driving heavy equipment. Soil compaction may extend down below the top few centimeters of the soil to greater depths. Soil crusting is a surface phenomenon usually caused by the soil fines rising to the surface of the soil due to kinetic energy, usually from water, acting on the soil. This segregation of the soil into an upper surface of fine particles and a lower layer of coarser-size particles results in the formation of a crust. The resulting crust tends to be a hard, impermeable superficial layer, reducing water penetration into the soil and slowing the rate of soil drying.

Soil crusting often occurs as a result of sprinkled irrigation where the impact force of water droplets strikes the soil surface causing significant physical displacement of the soil particles and giving rise to the separation of fine particles and coarse particles. When the compositions disclosed herein are placed on the soil before the planter press wheel is applied, soil crusting can be prevented.

### Formulations

In some embodiments, the inventive compositions are liquid formulations. Non-limiting examples of liquid formulations include suspension concentrations and oil dispersions. In other embodiments, the inventive compositions are solid formulations. Non-limiting examples of liquid formulations include freeze-dried powders and spray-dried powders.

In a further aspect, the compositions can comprise a wetting agent or dispersant, a binder or adherent, an aqueous solvent and/or a non-aqueous co-solvent. The compositions provided herein can be formulated as a solid; as a powder, lyophilizate, pellet or granules; as a liquid or gel; or as an emulsion, colloid, suspension or solution.

Compositions of the present invention may include formulation inerts added to compositions comprising cells, cell-free preparations or metabolites to improve efficacy, stability, and usability and/or to facilitate processing, packaging and end-use application. Such formulation inerts and ingredients may include carriers, stabilization agents, nutrients, or physical property modifying agents, which may be added individually or in combination. In some embodiments, the carriers may include liquid materials such as water, oil, and other organic or inorganic solvents and solid materials such as minerals, polymers, or polymer complexes derived biologically or by chemical synthesis. In some embodiments, the carrier is a binder or adhesive that facilitates adherence of the composition to a plant part, such as a seed or root. See, for example, Taylor, A. G., et al., “Concepts and Technologies of Selected Seed Treatments”, Annu. Rev. Phytopathol. 28: 321-339 (1990). The stabilization agents may include anti-caking agents, anti-oxidation agents, desiccants, protectants or preservatives. The nutrients may include carbon, nitrogen, and phosphors sources such as sugars, polysaccharides, oil, proteins, amino acids, fatty acids and phosphates. The physical property modifiers may include bulking agents, wetting agents, thickeners, pH modifiers, rheology modifiers, dispersants, adjuvants, surfactants, film-formers, hydrotropes, builders, antifreeze agents or colorants. In some embodiments, the composition comprising cells, cell-free preparation and/or metabolites produced by fermentation can be used directly with or without water as the diluent without any other formulation preparation. In a particular embodiment, a wetting agent, or a dispersant, is added to a fermentation solid, such as a freeze-dried or spray-dried powder. A wetting agent increases the spreading and penetrating properties, or a dispersant increases the dispersibility and solubility of the active ingredient (once diluted) when it is applied to surfaces. Exemplary wetting agents are known to those of skill in the art and include sulfosuccinates and derivatives, such as MULTIWET™ MO-70R (Croda Inc., Edison, N.J.); siloxanes such as BREAK-THRU® (Evonik, Germany); nonionic compounds, such as ATLOX™ 4894 (Croda Inc., Edison, N.J.); alkyl polyglucosides, such as TERWET® 3001 (Huntsman International LLC, The Woodlands, Tex.); C12-C14 alcohol ethoxylate, such as TERGITOL® 15-S-15 (The Dow Chemical Company, Midland, Mich.); phosphate esters, such as RHODAFAC® BG-510 (Rhodia, Inc.); and alkyl ether carboxylates, such as EMULSOGEN™ LS (Clariant Corporation, North Carolina).

The formulations or application forms in question preferably comprise auxiliaries, such as extenders, solvents, spontaneity promoters, carriers, emulsifiers, dispersants, frost protectants, biocides, thickeners and/or other auxiliaries, such as adjuvants, for example. An adjuvant in this context is a component which enhances the biological effect of the formulation, without the component itself having a biological effect. Examples of adjuvants are agents which promote the retention, spreading, attachment to the leaf surface, or penetration.

These formulations are produced in a known manner, for example by mixing the active compounds with auxiliaries such as, for example, extenders, solvents and/or solid carriers and/or further auxiliaries, such as, for example, surfactants.

Suitable for use as auxiliaries are substances which are suitable for imparting to the formulation of the active compound or the application forms prepared from these formulations (such as, e.g., usable crop protection agents, such as spray liquors or seed dressings) particular properties such as certain physical, technical and/or biological properties.

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulphones and sulphoxides (such as dimethyl sulphoxide).

If the extender used is water, it is also possible to employ, for example, organic solvents as auxiliary solvents. Essentially, suitable liquid solvents are: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and also their ethers and esters, ketones such as acetone, methyl ethyl ketone, methyl isobutyl ketone or cyclohexanone, strongly polar solvents such as dimethylformamide and dimethyl sulphoxide, and also water.

In principle it is possible to use all suitable solvents. Suitable solvents are, for example, aromatic hydrocarbons, such as xylene, toluene or alkylnaphthalenes, for example, chlorinated aromatic or aliphatic hydrocarbons, such as chlorobenzene, chloroethylene or methylene chloride, for example, aliphatic hydrocarbons, such as cyclohexane, for example, paraffins, petroleum fractions, mineral and vegetable oils, alcohols, such as methanol, ethanol, isopropanol, butanol or glycol, for example, and also their ethers and esters, ketones such as acetone, methyl ethyl ketone, methyl isobutyl ketone or cyclohexanone, for example, strongly polar solvents, such as dimethyl sulphoxide, and water.

All suitable carriers may in principle be used. Suitable carriers are in particular: for example, ammonium salts and ground natural minerals such as kaolins, clays, talc, chalk, quartz, attapulgite, montmorillonite or diatomaceous earth, and ground synthetic minerals, such as finely divided silica, alumina and natural or synthetic silicates, resins, waxes and/or solid fertilizers. Mixtures of such carriers may likewise be used. Carriers suitable for granules include the following: for example, crushed and fractionated natural minerals such as calcite, marble, pumice, sepiolite, dolomite, and also synthetic granules of inorganic and organic meals, and also granules of organic material such as sawdust, paper, coconut shells, maize cobs and tobacco stalks.

Liquefied gaseous extenders or solvents may also be used. Particularly suitable are those extenders or carriers which at standard temperature and under standard pressure are gaseous, examples being aerosol propellants, such as halogenated hydrocarbons, and also butane, propane, nitrogen and carbon dioxide.

Examples of emulsifiers and/or foam-formers, dispersants or wetting agents having ionic or nonionic properties, or mixtures of these surface-active substances, are salts of polyacrylic acid, salts of lignosulphonic acid, salts of phenolsulphonic acid or naphthalenesulphonic acid, polycondensates of ethylene oxide with fatty alcohols or with fatty acids or with fatty amines, with substituted phenols (preferably alkylphenols or arylphenols), salts of sulphosuccinic esters, taurine derivatives (preferably alkyltaurates), phosphoric esters of polyethoxylated alcohols or phenols, fatty acid esters of polyols, and derivatives of the compounds containing sulphates, sulphonates and phosphates, examples being alkylaryl polyglycol ethers, alkylsulphonates, alkyl sulphates, arylsulphonates, protein hydrolysates, lignin-sulphite waste liquors and methylcellulose. The presence of a surface-active substance is advantageous if one of the active compounds and/or one of the inert carriers is not soluble in water and if application takes place in water.

Further auxiliaries that may be present in the formulations and in the application forms derived from them include colorants such as inorganic pigments, examples being iron oxide, titanium oxide, Prussian Blue, and organic dyes, such as alizarin dyes, azo dyes and metal phthalocyanine dyes, and nutrients and trace nutrients, such as salts of iron, manganese, boron, copper, cobalt, molybdenum and zinc.

Stabilizers, such as low-temperature stabilizers, preservatives, antioxidants, light stabilizers or other agents which improve chemical and/or physical stability may also be present. Additionally present may be foam-formers or defoamers.

Furthermore, the formulations and application forms derived from them may also comprise, as additional auxiliaries, stickers such as carboxymethylcellulose, natural and synthetic polymers in powder, granule or latex form, such as gum arabic, polyvinyl alcohol, polyvinyl acetate, and also natural phospholipids, such as cephalins and lecithins, and synthetic phospholipids. Further possible auxiliaries include mineral and vegetable oils.

There may possibly be further auxiliaries present in the formulations and the application forms derived from them. Examples of such additives include fragrances, protective colloids, binders, adhesives, thickeners, thixotropic substances, penetrants, retention promoters, stabilizers, sequestrants, complexing agents, humectants and spreaders. Generally speaking, the active compounds may be combined with any solid or liquid additive commonly used for formulation purposes.

Suitable retention promoters include all those substances which reduce the dynamic surface tension, such as dioctyl sulphosuccinate, or increase the viscoelasticity, such as hydroxypropylguar polymers, for example.

Suitable penetrants in the present context include all those substances which are typically used in order to enhance the penetration of active agrochemical compounds into plants. Penetrants in this context are defined in that, from the (generally aqueous) application liquor and/or from the spray coating, they are able to penetrate the cuticle of the plant and thereby increase the mobility of the active compounds in the cuticle. This property can be determined using the method described in the literature (Baur et al., 1997, Pesticide Science 51, 131-152). Examples include alcohol alkoxylates such as coconut fatty ethoxylate (10) or isotridecyl ethoxylate (12), fatty acid esters such as rapeseed or soybean oil methyl esters, fatty amine alkoxylates such as tallowamine ethoxylate (15), or ammonium and/or phosphonium salts such as ammonium sulphate or diammonium hydrogen phosphate, for example.

In some embodiments, the plant growth promoting Gram-negative bacteria and the EPS are incorporated into one or more agriculturally acceptable carriers.

Compositions of the present disclosure may comprise any suitable agriculturally acceptable carrier(s), including, but not limited to, seed-compatible carriers, foliar-compatible carriers and soil-compatible carriers.

In some embodiments, compositions of the present disclosure comprise one or more liquid and/or gel carriers. For example, in some embodiments, compositions of the present disclosure comprise an aqueous solvent and/or a nonaqueous solvent.

In some embodiments, compositions of the present disclosure comprise one or more inorganic solvents, such as decane, dodecane, hexylether and nonane; one or more organic solvents, such as acetone, dichloromethane, ethanol, hexane, methanol, propan-2-ol and trichloroethylene; and/or water.

Non-limiting examples of liquid/gel carriers that may be useful in compositions of the present disclosure include oils (e.g., mineral oil, olive oil, peanut oil, soybean oil, sunflower oil), polyethylene glycols (e.g., PEG 200, PEG 300, PEG 400, etc.), propylene glycols (e.g., PPG-9, PPG-10, PPG-17, PPG-20, PPG-26, etc.), ethoxylated alcohols (e.g., TOMADOL® (Air Products and Chemicals, Inc., Allentown, Pa.), TERGITOL™ 15-S surfactants such as TERGITOL™ 15-S-9 (The Dow Chemical Company, Midland, Mich.), etc.), polysorbates (e.g. polysorbate 20, polysorbate 40, polysorbate 60, polysorbate 80, etc.), silicones (siloxanes, trisiloxanes, etc.) and combinations thereof.

Additional examples of solvents that may be included in compositions of the present disclosure may be found in BURGES, FORMULATION OF MICROBIAL BIOPESTICIDES: BENEFICIAL MICROORGANISMS, NEMATODES AND SEED TREATMENTS (Springer Science & Business Media) (2012); Inoue & Horikoshi, J. FERMENTATION BIOENG. 71(3):194 (1991).

In some embodiments, compositions of the present disclosure comprise one or more solid carriers. For example, in some embodiments, compositions of the present disclosure comprise one or more powders (e.g., wettable powders) and/or granules.

Non-limiting examples of solid carriers that may be useful in compositions of the present disclosure include clays (e.g., attapulgite clays, montmorillonite clay, etc.), peat-based powders and granules, freeze-dried powders, spray-dried powders, spray-freeze-dried powders and combinations thereof.

Additional examples of solid carriers that may be included in compositions of the present disclosure may be found in BURGES, FORMULATION OF MICROBIAL BIOPESTICIDES: BENEFICIAL MICROORGANISMS, NEMATODES AND SEED TREATMENTS (Springer Science & Business Media) (2012).

Carriers incorporated into compositions of the present disclosure may comprise a growth medium suitable for culturing one or more of the microorganisms in the composition. For example, in some embodiments, compositions of the present disclosure comprise Czapek-Dox medium, glycerol yeast extract, mannitol yeast extract, potato dextrose broth and/or YEM media.

Selection of appropriate carrier materials will depend on the intended application(s) and the microorganism(s) present in the composition. In some embodiments, the carrier material(s) will be selected to provide a composition in the form of a liquid, gel, slurry, or solid.

Carriers may be incorporated into compositions of the present disclosure in any suitable amount(s)/concentration(s). The absolute value of the carrier amount/concentration/dosage may be affected by factors such as the type, size and volume of material to which the composition will be applied, the type(s) of microorganisms in the composition, the number of microorganisms in the composition, the stability of the microorganisms in the composition and storage conditions (e.g., temperature, relative humidity, duration). Those skilled in the art will understand how to select an effective amount/concentration/dosage using routine dose-response experiments.

In some embodiments, compositions of the present disclosure comprise one or more commercial carriers used in accordance with the manufacturer's recommended amounts/concentrations.

### Biological Deposit of Parachlorella kessleri Accession No. NCMA 202103001

A Biological Deposit of Parachlorella kessleri Accession No. NCMA 202103001 was made at the Provasoli-Guillard National Center for Marine Algae and Microbiota—Bigelow Laboratory for Ocean Sciences, (NCMA, 60 Bigelow Drive, East Boothbay, Me. 04544 U.S.A.) on Mar. 3, 2021 under the provisions of the Budapest Treaty and assigned by the International Depositary Authority the accession number 202103001. Upon issuance of a patent, all restrictions upon the Deposit will be irrevocably removed, and the Deposit is intended to meet the requirements of 37 CFR §§ 1.801-1.809. The Deposit will be maintained in the depository for a period of 30 years, or 5 years after the last request, or for the effective, enforceable life of the patent, whichever is longer, and will be replaced if necessary during that period; and the requirements of 37 CFR §§ 1.801-1.809 are met.

The present invention is further illustrated by the following examples that should not be construed as limiting. The contents of all references, patents, and published patent applications cited throughout this application, as well as the Figures, are incorporated herein by reference in their entirety for all purposes.

### EXAMPLES

### Example 1. Taxonomic Identification of Parachlorella kessleri Accession No. NCMA 202103001

An environmental sample containing dry rice stubble was collected, and an individual Chlorella-like strain was isolated from the sample and cryopreserved. Taxonomic identification of this strain was performed by genome sequencing of the 18S rRNA gene and the internal transcribed spacers, ITS1 and ITS2.

DNA was isolated from the strain and PCR amplified with the following primers:

| | 18S rRNA Primers |
|---|---|
| (SEQ ID NO: 1) | |
| | 5′-AACCTGGTTGATCCTGCCAGT-3′ |
| (SEQ ID NO: 2) | |
| | 5′-GGGCATCACAGACCTG-3′ |
| | ITS1 and ITS2 Primers |
| (SEQ ID NO: 3) | |
| | 5′-TCCGTAGGTGAACCTGCGG -3′ |
| (SEQ ID NO: 4) | |
| | 5′-TCCTCCGCTTATTGATATGC-3′ |

Amplified genome regions were purified and validated with multiple rounds of PCR. Genomic barcoding of the 18S and ITS regions of DNA was performed using Sanger sequencing. Partial sequences were used for subsequent analysis and comparison when full sequences were unavailable.

The genus and species of the strain was identified using the NCBI Basic Local Alignment Search Tool (BLAST). This analysis identified the strain as Parachlorella kessleri (see Table 1 and Table 2). When the ITS regions of Parachlorella kessleri Accession No. NCMA 202103001 was compared with that of Parachlorella kessleri strain UTEX262, an 82% ITS percent identity was found suggesting the two strains belong to different subspecies.

TABLE 1
BLAST results with 18S rRNA gene sequences.

| | Max | Total | Query | E | % | |
|---|---|---|---|---|---|---|
| Description | Score | Score | Cover | Value | Ident. | Accession |
| Parachlorella sp. | 2658 | 2658 | 100% | 0.0 | 99.93 | LC473527.1 |
| BX1.5 genes for 18S | | | | | | |
| rRNA, ITS1, 5.8S | | | | | | |
| rRNA, ITS2, 28S | | | | | | |
| rRNA, partial and | | | | | | |
| complete sequence | | | | | | |
| Parachlorella kessleri | 2656 | 2656 | 99% | 0.0 | 99.93 | AB162911.1 |
| genes for 18S rRNA, | | | | | | |
| ITS1, 5.8S rRNA, | | | | | | |
| ITS2 | | | | | | |
| Parachlorella kessleri | 2656 | 2656 | 99% | 0.0 | 99.93 | AB080309.1 |
| gene for 18S rRNA | | | | | | |
| partial sequence | | | | | | |

TABLE 2
BLAST results with ITS1 and ITS2 gene sequences.

| | Max | Total | Query | E | % | |
|---|---|---|---|---|---|---|
| Description | Score | Score | Cover | Value | Ident. | Accession |
| Parachlorella kessleri strain | 715 | 715 | 56% | 0.0 | 98.75 | KF163441.1 |
| MMPBKK-1 5.8S | | | | | | |
| ribosomal RNA gene, | | | | | | |
| partial sequence, internal | | | | | | |
| transcribed spacer 2 | | | | | | |
| Parachlorella kessleri strain | 483 | 483 | 99% | 7.00E−132 | 98.55 | AY323477.1 |
| SAG 211-11g internal | | | | | | |
| transcribed spacer 2 | | | | | | |
| Parachlorella kessleri strain | 920 | 1139 | 92% | 0.0 | 97.35 | KX021360.1 |
| TY02 18S ribosomal RNA | | | | | | |
| gene, partial sequence; | | | | | | |
| internal transcribed spacer 1 | | | | | | |

### Example 2. Analysis of Glycosyl Composition with EPS from Parachlorella kessleri Accession No. NCMA 202103001

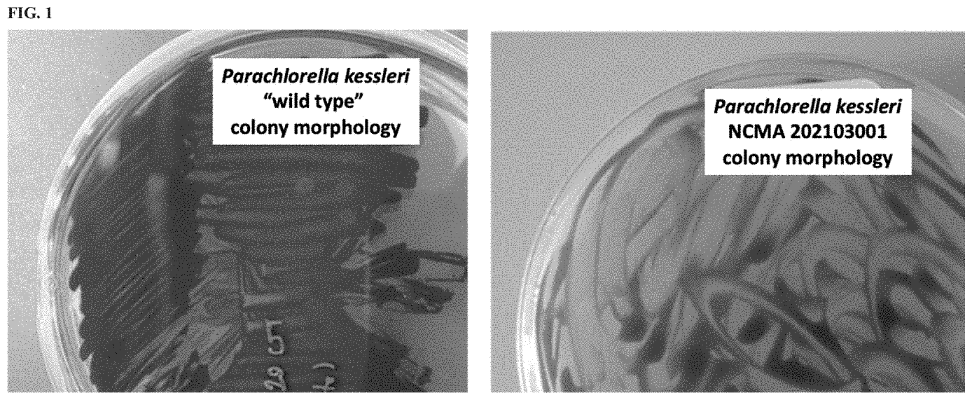

When Parachlorella kessleri Accession No. NCMA 202103001 was streaked onto agar plates containing BG-11 medium (ATCC Medium 616), the resulting colony morphology differed from wild-type Parachlorella kessleri in its texture and firmness (see FIG. 1). Parachlorella kessleri Accession No. NCMA 202103001 grown in liquid culture demonstrated rapid growth and a highly viscous biomass suggesting an unusual ability to produce EPS.

A method for purification of EPS was developed involving the following steps:
1. Dilution and centrifugation to separate algal biomass from the supernatant;
2. A filtration or clarification step as necessary to remove remaining biomass from the supernatant;
3. Concentration of supernatant via vaporization or Tangential Flow Filtration (TFF);
4. Isopropanol (IPA) precipitation of EPS from the concentrated supernatant; and
5. Lyophilization of precipitated EPS.
   This purification process was used to isolate EPS for further characterization.

EPS from Parachlorella kessleri Accession No. NCMA 202103001 was diluted to 1 mg/ml and analyzed via HPLC on a size exclusion column to determine its molecular weight. A SUPEROSE® 6 column (GE Life Sciences) was used for separation of analytes that were detected by refractive index (RI) and ultraviolet (UV) absorption at 280 nm. Molecular weight standards having sizes of 40 kilodaltons (kD), 167 kD, and 511 kD were run under the same conditions to allow calculation of the molecular weight of the EPS.

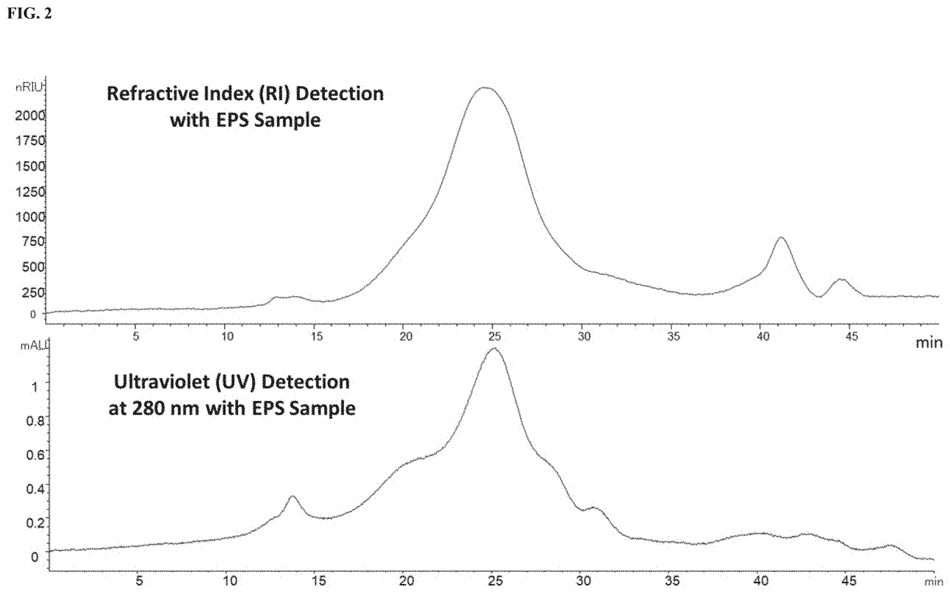

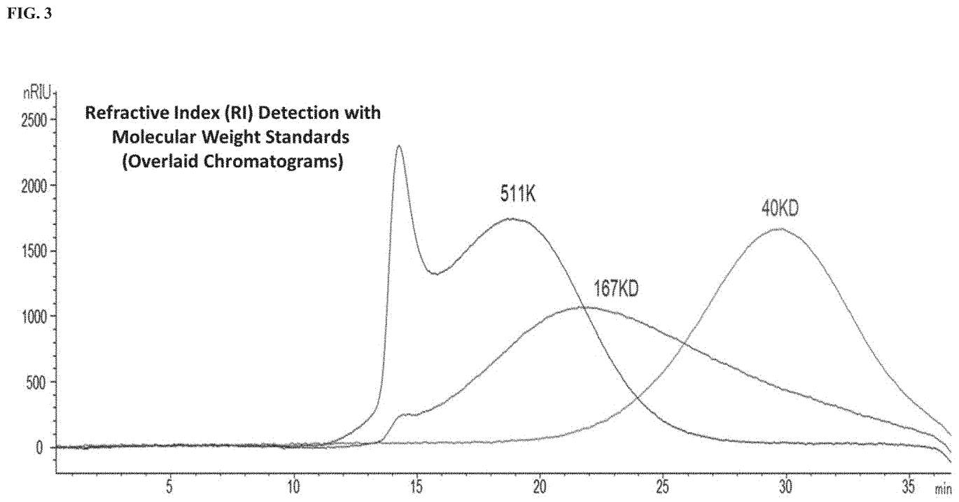

FIG. 2 shows the Parachlorella kessleri Accession No. NCMA 202103001 EPS to have one dominant peak centered at about 25 minutes using RI detection. Based on the elution times of the molecular weight standards using RI detection (FIG. 3), the size of the EPS was calculated to be about 120 kD. The UV chromatogram of the EPS also showed a peak centered around 25 minutes indicating that the main peak contains some non-carbohydrate material (e.g., protein) (see FIG. 2).

A glycosyl composition analysis of the Parachlorella kessleri Accession No. NCMA 202103001 EPS was performed by combined gas chromatography/mass spectrometry (GC-MS) of the per-O-trimethylsilyl (TMS) derivatives of the monosaccharide methyl glycosides. The TMS derivatives were produced from the EPS by acidic methanolysis as described in Santander et al. (2013) Microbiology 159:1471. Glycosyl composition analysis was then performed by GC-MS of the alditol acetates (AAs) as described by Pella et al. (2012) Methods Enzymol. 510:121.

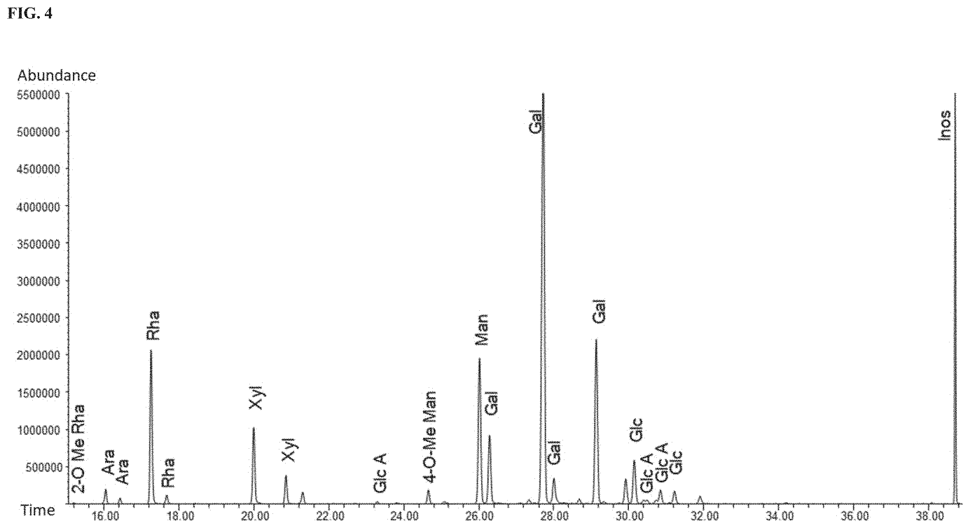

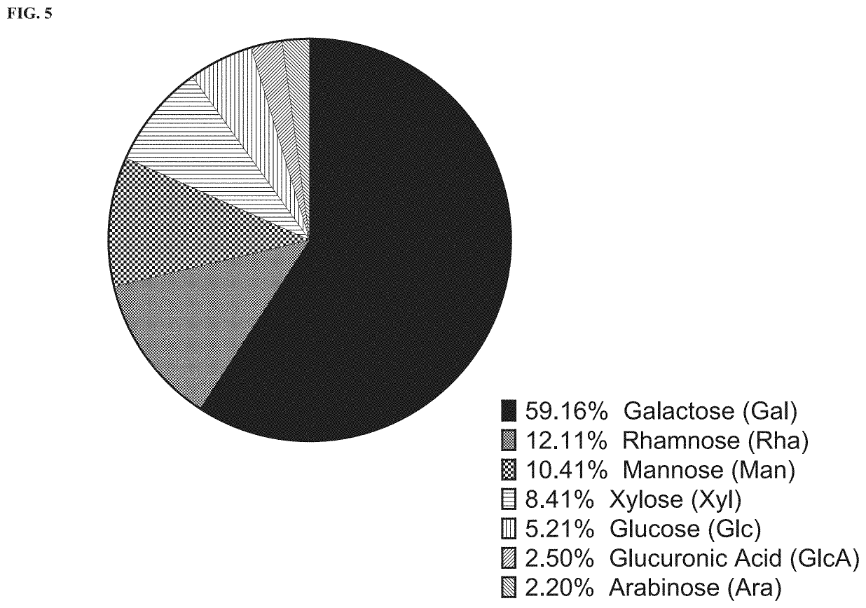

The glycosyl composition analysis identified several monosaccharides with galactose being the principal component followed by mannose, xylose, rhamnose, and small amounts of glucuronic acid and arabinose (see Table 3 and FIGS. 4-5). Several peaks were observed that suggested the presence of methylated residues. To verify the presence of methylated sugars, an AA of the EPS was run. The results showed that the EPS contains trace amounts of 2-methyl rhamnose and 4-methyl mannose. The percentage of carbohydrate in the EPS sample was determined to be about 72%.

TABLE 3
Quantification of monosaccharides detected in the Parachlorella kessleri
Accession No. NCMA 202103001 EPS.

| Glycosyl residue | Mass (μg) | Mol % |
|---|---|---|
| Galactose (Gal) | 157.4 | 59.1 |
| Rhamnose (Rha) | 29.4 | 12.1 |
| Mannose (Man) | 27.7 | 10.4 |
| Xylose (Xyl) | 18.7 | 8.4 |
| Glucose (Glc) | 13.9 | 5.2 |
| Glucuronic Acid (GlcA) | 7.2 | 2.5 |
| Arabinose (Ara) | 4.9 | 2.2 |
| Ribose (Rib) | n.d. | — |
| Fucose (Fuc) | n.d. | — |
| Galacturonic acid (GalA) | n.d. | — |
| N-Acetyl Galactosamine (GalNAc) | n.d. | — |
| N-Acetyl Glucosamine (GlcNAc) | n.d. | — |
| N-Acetyl Mannosamine (ManNAc) | n.d. | — |
| Σ= | 259.2 | |

### Example 3. Analysis of Glycosyl Linkage with EPS from Parachlorella kessleri Accession No. NCMA 202103001

A glycosyl linkage analysis was also conducted with the Parachlorella kessleri Accession No. NCMA 202103001 EPS. The EPS samples were carboxyl methylated, reduced, permethylated, hydrolyzed, reduced and acetylated. The resulting partially methylated alditol acetates (PMAAs) analyzed by GC-MS. The procedure generally followed that described by Willis et al. (2013) PNAS, 110 (19) 7868-7873.

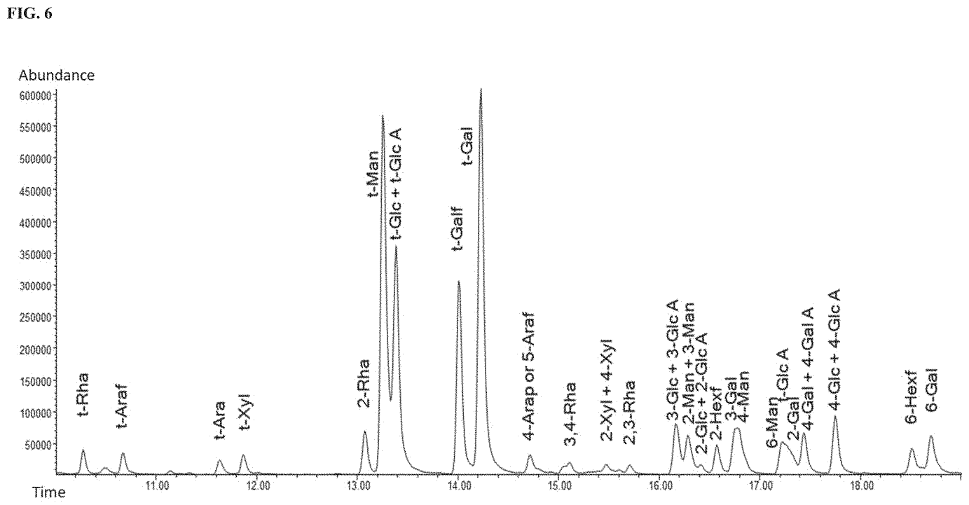

The linkage data were generally consistent with the composition analysis showing galactose residues as the main component of the EPS with significant amounts of other hexose residues (see Table 4 and FIG. 6). A relatively large percentage of the EPS was represented as terminal residues indicating that along with the major polysaccharide in FIG. 2, the smaller molecular weight peaks seen in the chromatogram are likely monosaccharides. The number of non-terminal linkages is relatively large with no one linked residue predominating, which is characteristic of a polysaccharide with many different linkages as opposed to repeating oligosaccharides.

TABLE 4
Relative percentage of each detected glycosyl linkage in the
Parachlorella kessleri Accession No. NCMA 202103001 EPS.

| | Relative |
|---|---|
| Glycosyl Residue | Area % |
| Terminal Galactopyranosyl residue (t-Gal) | 21.7 |
| Terminal Mannopyranosyl residue (t-Man) | 19.5 |
| Terminal Glucopyranosyluronic acid residue (t-Glc A) | 10.8 |
| Terminal Galactofuranosyl residue (t-Galf) | 10.5 |
| Terminal Glucopyranosyl residue (t-Glc) | 3.2 |
| 6 linked Galactopyranosyl residue (6-Gal) | 2.9 |
| 2 linked Rhamnopyranosyl residue (2-Rha) | 2.4 |
| 4 linked Galactopyranosyl residue (4-Gal) | 2.3 |
| 3 linked Glucopyranosyl residue (3-Glc) | 2 |
| 4 linked Mannopyranosyl residue (4-Man) | 2 |
| 3 linked Galactopyranosyl residue (3-Gal) | 1.8 |
| 4 linked Glucopyranosyl residue (4-Glc) | 1.8 |
| 3 linked Mannopyranosyl residue (3-Man) | 1.7 |
| 4 linked Glucopyranosyluronic Acid residue (4-Glc A) | 1.7 |
| 6 linked Hexofuranosyl residue (6-Hexf) | 1.7 |
| 2 linked Hexofuranosyl residue (2-Hexf) | 1.6 |
| 6 linked Mannopyranosyl residue (6-Man) | 1.3 |
| Terminal Rhamnopyranosyl residue (t-Rha) | 1.2 |
| 4 linked Arabinopyranosyl residue or 5 linked | 1.2 |
| Arabinofuranosyl residue (4-Arap or 5-Araf) | |
| 3 linked Glucopyranosyluronic Acid residue (3-Glc A) | 1.2 |
| Terminal Arabinofuranosyl residue (t-Araf) | 1.1 |
| Terminal Xylopyranosyl residue (t-Xyl) | 1.1 |
| 2 linked Mannopyranosyl residue (2-Man) | 1.1 |
| Terminal Arabinopyranosyl residue (t-Ara) | 0.9 |
| 2 linked Galactopyranosyl residue (2-Gal) | 0.9 |
| 3,4 linked Rhamnopyranosyl residue (3,4-Rha) | 0.6 |
| 2,3 linked Rhamnopyranosyl residue (2,3-Rha) | 0.5 |
| 4 linked Xylopyranosyl residue (4-Xyl) | 0.4 |
| 2 linked Glucopyranosyluronic Acid residue (2-Glc A) | 0.3 |
| 4 linked Galactopyranosyluronic Acid residue (4-Gal A) | 0.3 |
| 2 linked Glucopyranosyl residue (2-Glc) | 0.2 |
| 2 linked Xylopyranosyl residue (2-Xyl) | 0.1 |

### Example 4. Rheological Profiling of EPS from Parachlorella kessleri Accession No. NCMA 202103001

Several analyses were performed to further characterize the EPS from Parachlorella kessleri Accession No. NCMA 202103001. The rheological profiling analyses capture typical behaviors of a “structured liquid” including: modulus (i.e., rigidity), yield stress (i.e., strength), and viscosity across a range of shear conditions and zero shear viscosity to characterize “at-rest” and storage conditions.

The EPS was prepared for testing by mixing 0.5 g of the EPS powder with 50 ml of DI water which was held at 50° C. while constantly stirring. Once visually homogenous, the sample was allowed to cool to room temperature before performing the testing.

Testing was performed on a Discovery Hybrid Rheometer-2 (DHR-2) (TA Instruments) fitted with a 40 mm sand blasted plate-plate geometry set at a 500 micrometer test gap. A solvent trap cover was employed for all testing to minimize drying of the sample at the exposed edges.

### Viscosity/Shear Profiling

Viscosity/shear profiling entails subjecting a material to a range of shear conditions and observing its viscosity throughout. From the resulting “flow curve” viscosity at any relevant shear rates or stresses and the degree of non-Newtonian (typically shear thinning) behavior exhibited by a material can be identified and quantified.

Controlled rate viscosity profiles, where shear rate is swept, typically across mid to high shear rates, are good for obtaining a rapid viscosity profile to correlate to a range of handling conditions, particularly where a material is forced to flow at certain rates through the action of pumps, coating equipment or manually applied forces.

A shear rate sweep test was performed with the EPS from Parachlorella kessleri Accession No. NCMA 202103001. This test applies a wide range of shear rates to the sample to quantify viscosity under different flow conditions. This test also has the potential to capture zero shear viscosity, the plateau viscosity seen when under very low stress conditions.

Following a 30s equilibration time at 25° C. the samples were exposed to a 30s pre shear at a rate of 1s⁻¹, this led immediately into a shear rate sweep from 1s⁻¹ to 1000s⁻¹, logarithmically spaced, 8 points per decade of shear rate. Shear rate applied for 30s at each point and the viscosity averaged over the final 5s.

The viscosity of the EPS at the highest and lowest shear rates applied is presented in Table 5. The EPS displayed non-Newtonian shear thinning behavior over the full range of shear rates applied.

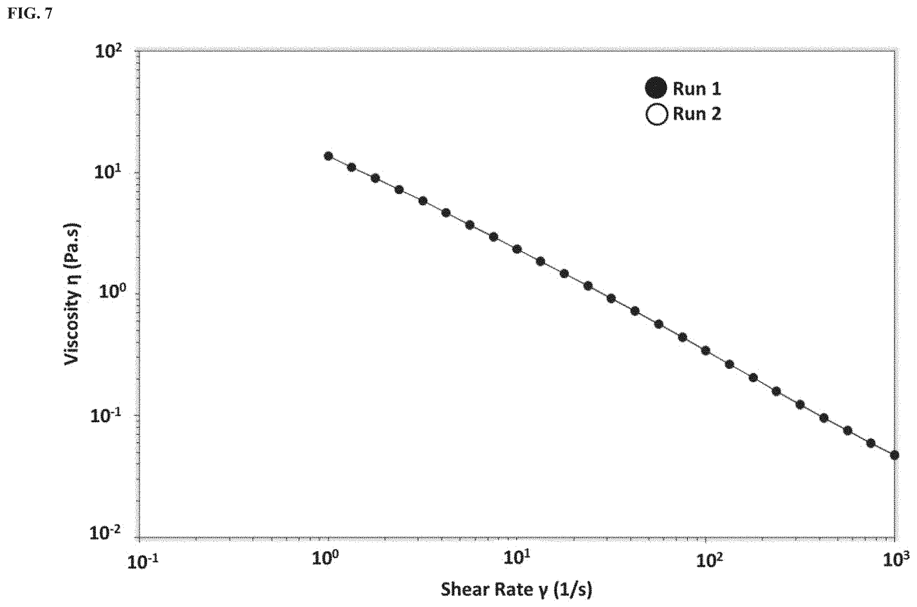

TABLE 5
Viscosity of the EPS at the highest and lowest shear rates applied in FIG. 7.

| | Viscosity (mPa.s) at 1 s⁻¹ | | | Viscosity (mPa.s) at 1000 s⁻¹ | | |
|---|---|---|---|---|---|---|
| Sample | Run 1 | Run 2 | Mean | Run 1 | Run 2 | Mean |
| EPS from Parachlorella | 13700 | 13700 | 13700 | 47.8 | 47.1 | 47.5 |
| kessleri Accession No. NCMA | | | | | | |
| 202103001 | | | | | | |

### Controlled Stress Viscosity Profiles

These are usually gentle tests where the sample is subjected to incrementing stresses that initially result in “creeping” flow where the sample's plateau zero shear viscosity prevails. As the stress is increased shear thinning—or in the case of extreme shear thinning: “yielding”—ensues and the sample's viscosity decreases towards an often much lower infinite-shear viscosity. Zero shear viscosity is the plateau viscosity often seen as shear rate is reduced towards zero. It serves, therefore, as a measure of the effective viscosity in an at-rest condition. Because of this, zero shear viscosity of a multi-phase formulation—and more significantly, of the continuous phase in such a system—is a significant contributor to stability against sedimentation or creaming, a high value resisting movement of the dispersed phase.

A shear stress sweep test was performed with the EPS from Parachlorella kessleri Accession No. NCMA 202103001. This test measures viscosity under controlled stress conditions and takes advantage of the accurate stress control of the rheometer to focus on quantifying the zero shear viscosity plateau.

Following a 60s equilibration time at 25° C. the samples were subjected to a shear stress up-sweep from 0.1 Pa to 100 Pa, logarithmically spaced, 8 points per decade of shear stress. Steady-state sensing was employed to ensure individual viscosity readings had reached an acceptable degree of elastic or thixotropic equilibrium before recording a reading. At each step of the test, viscosity was monitored every 10 seconds. Viscosity was recorded only when 3 successive measurements were within 5% of each other. A 60s timeout was set, meaning that if an equilibrium viscosity was not achieved after that time the viscosity at that instant was recorded regardless of degree of equilibrium.

The zero shear viscosity of the EPS is shown in Table 6. The controlled shear stress sweep testing clearly identified the zero shear viscosity plateau at the lowest stresses applied.

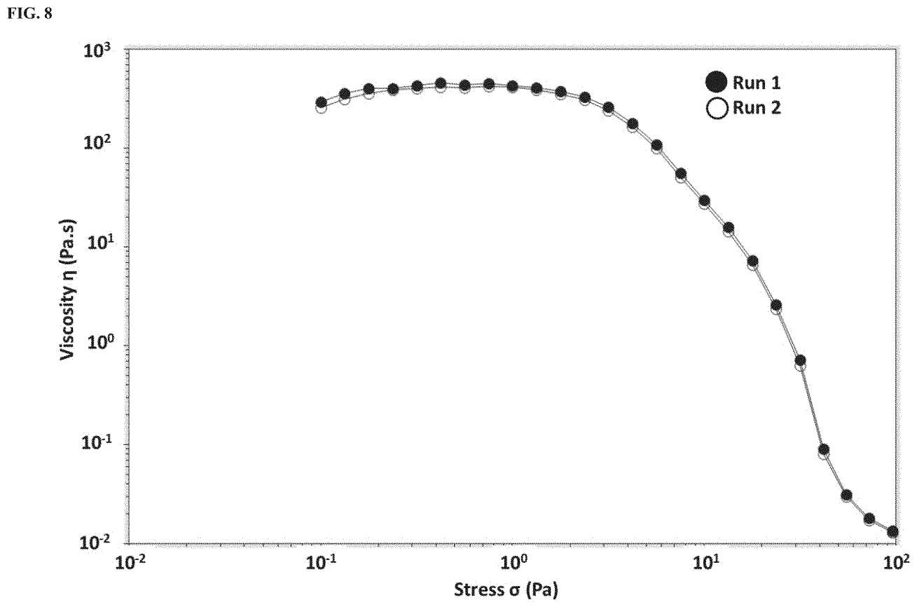

TABLE 6
Zero shear viscosity of the EPS quantified as
the average of the plateau observed in FIG. 8

| | Zero Shear Viscosity (mPa · s) | | |
|---|---|---|---|
| Sample | Run 1 | Run 2 | Mean |
| EPS from Parachlorella kessleri | 430000 | 407000 | 418000 |
| Accession No. NCMA 202103001 | | | |

### Oscillation Stress Sweep

The oscillation stress sweep test provides a simple quantification of the rigidity and strength of soft solid structure present throughout a sample. The test entails the application of small, incrementing sinusoidal (i.e., clockwise then counterclockwise) shear stresses to the sample while monitoring its resulting deformation and/or flow. In the early stages of the test, the stress is sufficiently low to preserve structure. The presence of this structure is revealed by dominant elastic deformation (rather than viscous flow) indicated by a phase angle plateau at low values. Phase angle is a measure of the relative dominance of elastic or viscous response of the sample and ranges from 0° for an ideal elastic material (i.e., a perfect solid) to 90° for an ideal viscous material (a perfect liquid). At this stage, the sample rigidity, the complex modulus, also remains at a plateau value. As the test progresses, the incrementing applied stress eventually disrupts sample structure as the yielding process progresses. This is manifested as a loss of elastic response (phase angle rises) and an accompanying decrease in rigidity (complex modulus decreases).

An oscillation stress sweep test was performed to identify the presence and strength of soft solid structure when the EPS was under low stress conditions. Results from this test are thought to be of relevance to handling and first touch properties.

Following a 60s equilibration time at 25° C. the samples were exposed to an oscillatory stress sweep ranging from 0.01 Pa to 1000 Pa, 10 points per decade, 1 Hz oscillation frequency. A step termination was set such that if the oscillation strain exceeded 1500% at any point, then the test would end prematurely.

The structural metrics determined from this testing are presented in Table 7. The oscillation stress sweep testing revealed clearly structured behavior under low stress conditions that yielded to viscous flow once the stress exceeds a certain yielding value.

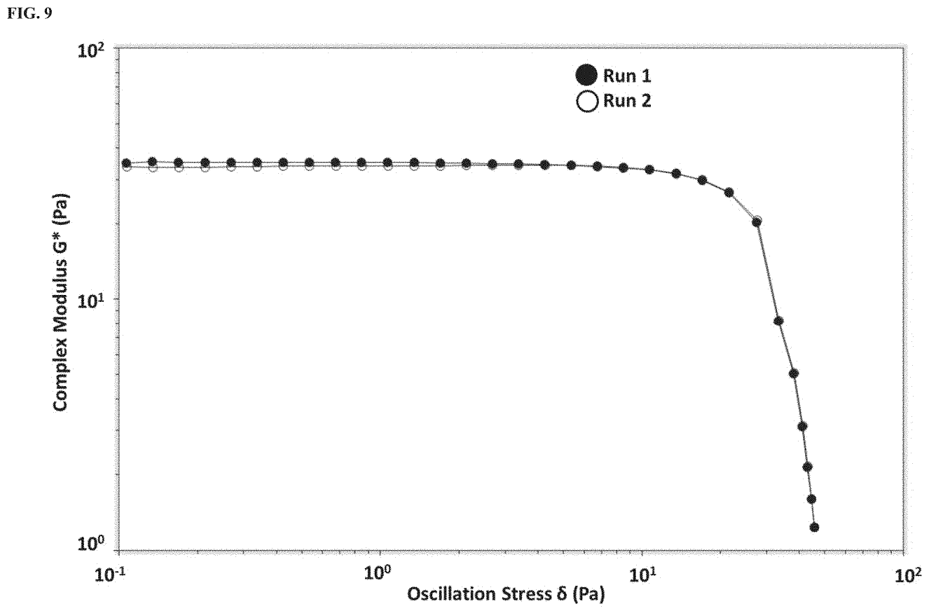

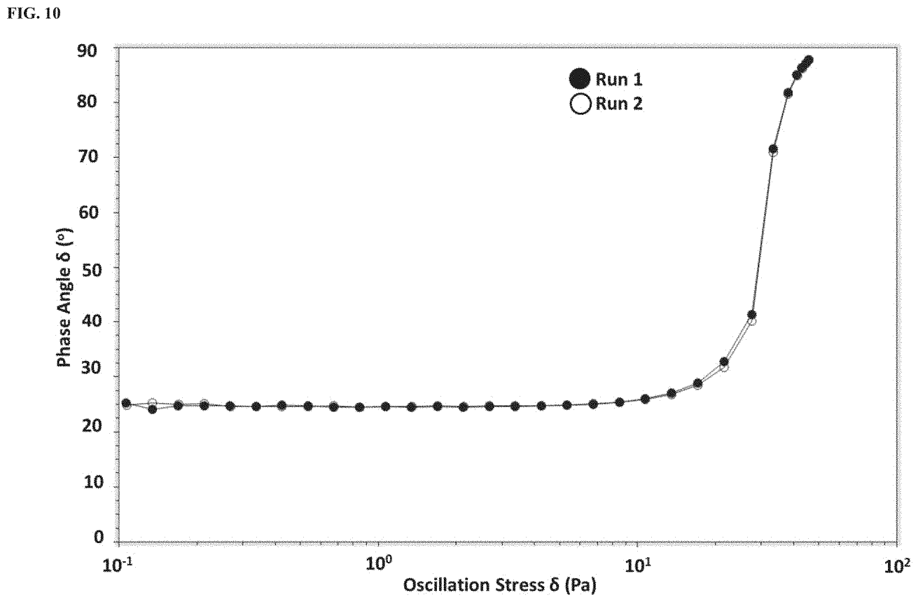

TABLE 7
Structural metrics of the EPS taken from the oscillation stress sweep data displayed
in FIGS. 9-10

| | Complex Modulus Plateau (Pa) | | | Phase Angle Plateau (°) | | |
|---|---|---|---|---|---|---|
| Sample | Run 1 | Run 2 | Mean | Run 1 | Run 2 | Mean |
| EPS from Parachlorella | 35.1 | 33.8 | 34.4 | 24.6 | 24.8 | 24.7 |
| kessleri Accession No. | | | | | | |
| NCMA 202103001 | | | | | | |

### Example 5. Comparison of Rheological Properties of EPS from Parachlorella kessleri Accession No. NCMA 202103001, Xanthan Gum, and EPS from Porphyridium cruentum UTEX 161

The testing and analyses performed in Example 4 were repeated with the common rheology modifier, xanthan gum, and EPS from Porphyridium cruentum UTEX 161 to benchmark and compare the rheological properties of the EPS from Parachlorella kessleri Accession No. NCMA 202103001 with those of xanthan gum and EPS from Porphyridium cruentum UTEX 161.

The xanthan gum sample was prepared for testing by mixing 1 g of the powder with 99 g of DI water using a stirrer bar at a low speed for 10 minutes. Testing was performed on a research rheometer (AR2000, TA Instruments) fitted with a 40 mm sand blasted plate-plate geometry set to a 500 μm test gap. A solvent trap cover was employed for all testing to minimize drying of the sample at the exposed edges.

The EPS sample from Porphyridium cruentum UTEX 161 was prepared for testing by first crushing the sample to a fine powder in a pestle and mortar, then mixing 0.2 g of the powder with 19.8 g of DI water (previously heated to 35° C.) using a stirrer bar at a low speed for 1.5 hours. This extended mixing period was required due to the difficulty of avoiding clumping during addition. The sample was then allowed to hydrate overnight prior to analysis. Testing was performed on a research rheometer (DHR-10, TA Instruments) fitted with a 40 mm sand blasted plate-plate geometry to a 500 μm test gap. A solvent trap cover was employed for all testing to minimize drying of the sample at the exposed edges.

### Controlled Stress Viscosity Profiles

A shear stress sweep test was performed with xanthan gum and the EPS from Porphyridium cruentum UTEX 161—for comparison with the results noted in Example 4 for the EPS from Parachlorella kessleri Accession No. NCMA 202103001. This test measures viscosity under controlled stress conditions and takes advantage of the accurate stress control of the rheometer to focus on quantifying the zero shear viscosity plateau.

Following a 120s equilibration time at 20-25° C. (20° C. for xanthan gum; 25° C. for the EPS from Porphyridium cruentum UTEX 161), the samples were subjected to a shear stress sweep from 0.1 Pa to 100 Pa, logarithmically spaced, 8 points per decade of shear stress, each stress applied for 30s with the viscosity averaged over the final 5s of each step.

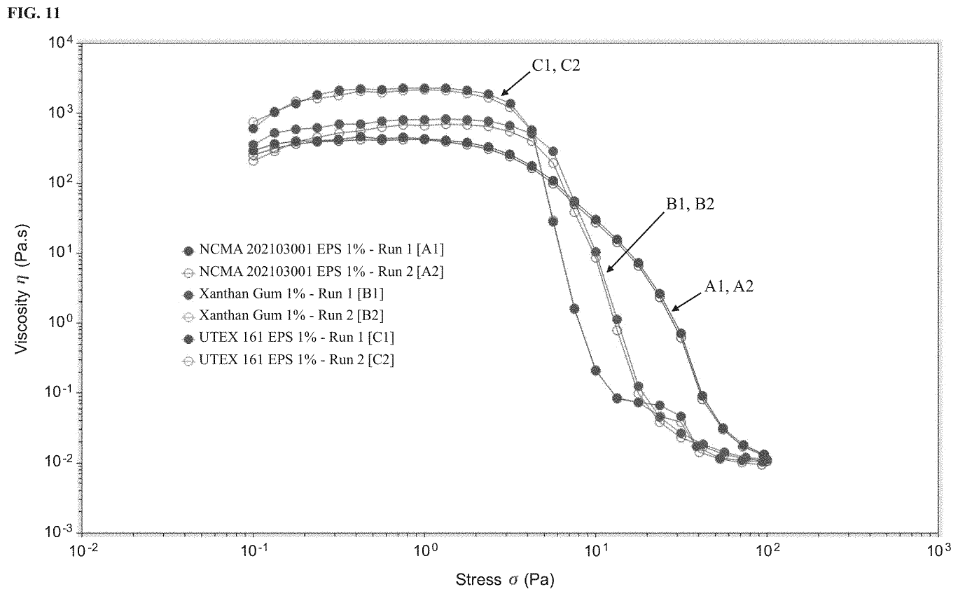

The zero shear viscosities of the EPS from Parachlorella kessleri Accession No. NCMA 202103001, xanthan gum, and the EPS from Porphyridium cruentum UTEX 161 are shown in Table 8. According to the results of the shear stress sweeps, depicted in FIG. 11, the xanthan gum exhibited a higher zero shear viscosity plateau than the EPS from Parachlorella kessleri Accession No. NCMA 202103001, and the EPS from Porphyridium cruentum UTEX 161 exhibited a significantly higher zero shear viscosity plateau than both the EPS from Parachlorella kessleri Accession No. NCMA 202103001 and the xanthan gum.

TABLE 8
Zero shear viscosities of the EPS from Parachlorella kessleri
Accession No. NCMA 202103001, xanthan gum, and the EPS from
Porphyridium cruentum UTEX 161, quantified as the average
of the plateaus observed in FIG. 11.

| | Zero Shear Viscosity (mPa · s) | | |
|---|---|---|---|
| Sample | Run 1 | Run 2 | Mean |
| EPS from Parachlorella kessleri | 430 | 407 | 418 |
| Accession No. NCMA 202103001 - 1% | | | |
| Xanthan Gum - 1% | 802 | 677 | 740 |
| EPS from Porphyridium | 2222 | 2034 | 2128 |
| cruentum UTEX 161 - 1% | | | |

### Oscillation Stress Sweep

In addition to the shear stress sweep test, an oscillation stress sweep test was performed with each of the additional samples to identify the presence and strength of soft solid structure when the samples were under low stress conditions. Results from this test are thought to be of relevance to handling and first touch properties.

Following a 120s equilibration time at 20-25° C. (20° C. for xanthan gum; 25° C. for the EPS from Porphyridium cruentum UTEX 161), the samples were exposed to an oscillatory stress sweep ranging from 0.1 Pa to 1000 Pa, 10 points per decade, 1 Hz oscillation frequency. A step termination was set such that if the oscillation strain exceeded 1500% at any point, then the test would end prematurely.

The structural metrics determined from this testing are presented in Table 9. The results for the EPS from Parachlorella kessleri Accession No. NCMA 202103001 show a more rigid (i.e., higher complex modulus plateau) and stronger (i.e., higher yield stress) structure compared to the xanthan gum. The results for the EPS from Porphyridium cruentum UTEX 161 exhibit a complex modulus plateau in a very similar range to that of the EPS from Parachlorella kessleri Accession No. NCMA 202103001. However, the yield stress of the EPS from Porphyridium cruentum UTEX 161 is much lower than the EPS from Parachlorella kessleri Accession No. NCMA 202103001 and the xanthan gum, indicating a weaker structure.

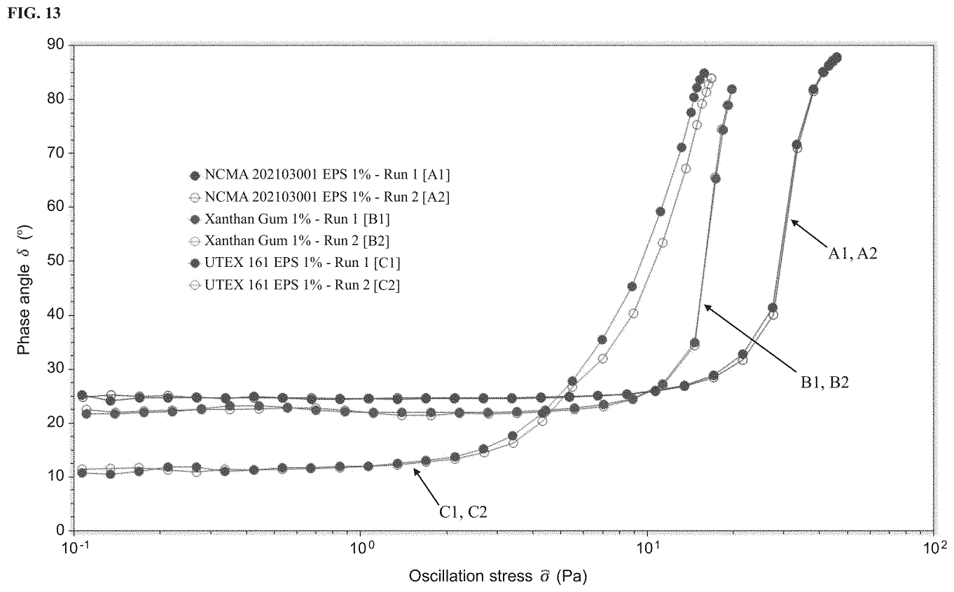

Yield stress was quantified using an onset point model fit to the phase angle data in FIG. 13. This entailed fitting one straight line through the low stress plateau and a second line through the inflection point as the sample yields. The stress at which these two lines cross was taken as the yield stress.

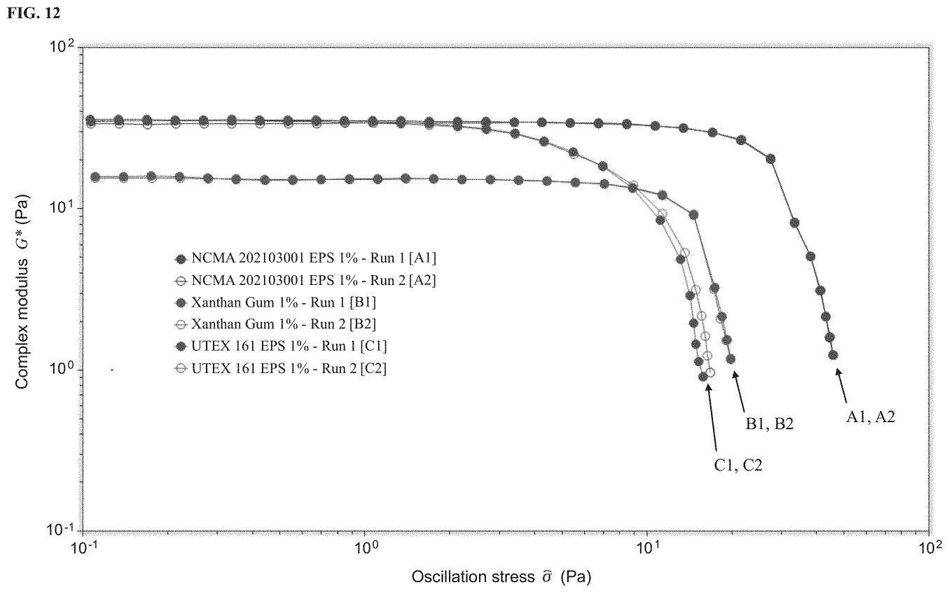

TABLE 9
Structural metrics of the EPS from Parachlorella kessleri Accession No. NCMA
202103001, xanthan gum, and the EPS from Porphyridium cruentum UTEX 161 taken from
the oscillation stress sweep data displayed in FIGS. 12-13.

| | Complex Modulus Plateau | | | | | | | | |
|---|---|---|---|---|---|---|---|---|---|
| | (Pa) | | | Phase Angle Plateau (Pa) | | | Yield Stress (Pa) | | |
| Sample | Run 1 | Run 2 | Mean | Run 1 | Run 2 | Mean | Run 1 | Run 2 | Mean |
| EPS from | 35.1 | 33.8 | 34.4 | 24.6 | 24.8 | 24.7 | 25.2 | 24.3 | 24.8 |
| Parachlorella | | | | | | | | | |
| kessleri | | | | | | | | | |
| Accession No. | | | | | | | | | |
| NCMA | | | | | | | | | |
| 202103001-1% | | | | | | | | | |
| Xanthan Gum- | 15.3 | 15.4 | 15.4 | 22.2 | 22.3 | 22.3 | 13.7 | 13.6 | 13.7 |
| 1% | | | | | | | | | |
| EPS from | 35.5 | 34.7 | 35.1 | 11.6 | 11.6 | 11.6 | 7.64 | 6.74 | 7.19 |
| Porphyridium | | | | | | | | | |
| cruentum UTEX | | | | | | | | | |
| 161-1% | | | | | | | | | |

### Example 6. Effects of Parachlorella kessleri Accession No. NCMA 202103001 Biomass and EPS on Soil and Soil Microbes

Sand soil samples were collected from Douglas, Ga. The following treatments were applied to the soil samples: 1) live-cell crude biomass; 2) live-cell washed biomass; 3) supernatant; 4) lysed crude biomass; 5) lysed washed biomass; 6) PHYCOTERRA® (whole cell Chlorella microalgae) (“PT”); and 7) untreated control (“UTC”). Treatments 1) to 5) were each with Parachlorella kessleri Accession No. NCMA 202103001. The supernatant contained EPS separated from cells of Parachlorella kessleri Accession No. NCMA 202103001.

### Water Holding Capacity

A water holding apparatus was used to measure the water holding capacity of the different soil samples. The following masses were determined in the apparatus: 1) the mass of the dry soil sample before applying water; 2) the mass of the wet soil after a volume of water was applied and allowed to drain; and 3) the mass of the retained water (i.e., the difference between the previous two masses). The water holding capacity was determined as a percentage representing the mass of the retained water divided by the mass of the soil sample.

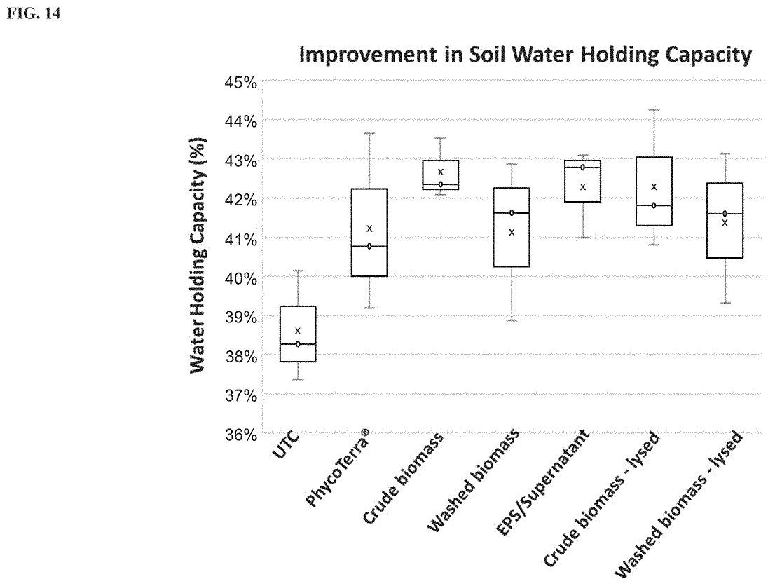

Each treatment containing microalgal biomass increased the water holding capacity of the Georgia soil samples compared to the untreated control sample (see FIG. 14). Surprisingly, the supernatant containing Parachlorella kessleri Accession No. NCMA 202103001 EPS also significantly increased water holding capacity (see “EPS/Supernatant” in FIG. 14).

### Dry Soil Aggregate Size

Dry soil aggregate size distribution of each of the soil samples was analyzed through use of a stacked sieve assay with each level of sieve containing a different sized mesh screen. A dry soil sample of approximately 50 g (exact weight obtained) was poured through a 4 mm sieve. Soil that passed through that sieve was then added to the top sieve pan in a set of 5 stacked pans, with each layer of the stack having progressively smaller screens and the bottom pan being a collection pan (i.e., 5 layers: 2 mm sieve, 1 mm sieve, 0.5 mm sieve, 0.25 mm sieve, and catch pan). The stack of sieves was then shaken on an orbital shaker for 5 minutes. Larger aggregates were caught in the higher sieves with only dust passing through to the bottom catch pan. The amount of material caught on each sieve level was then weighed and a simple calculation used to determine the mean size of dry soil aggregates in each sample.

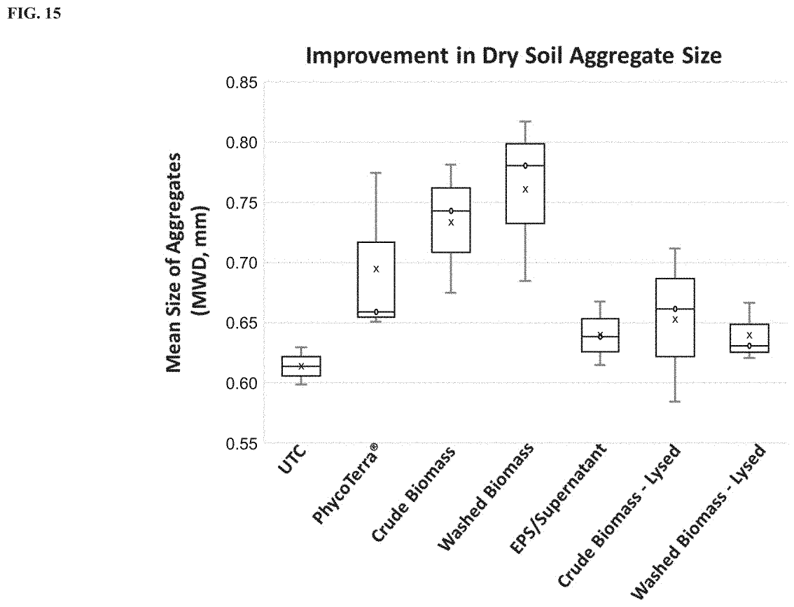

The crude and washed biomass of Parachlorella kessleri Accession No. NCMA 202103001 and PHYCOTERRA® (whole cell Chlorella microalgae) each increased the mean size of the dry soil aggregates compared to the untreated control soil whereas the supernatant containing the EPS and the lysed biomass samples from Parachlorella kessleri Accession No. NCMA 202103001 had no significant effect on dry soil aggregate size with the sand soil from Douglas, Ga. (see FIG. 15).

### Culturable Bacterial Populations

Culturable bacterial populations were counted 7 days after treatment by preparing a series of 10-fold dilutions of soil samples before applying them to Petri dishes containing an agar-based medium and counting the resulting colonies. Untreated soil samples were also diluted and applied to the Petri dishes and counted for comparison.

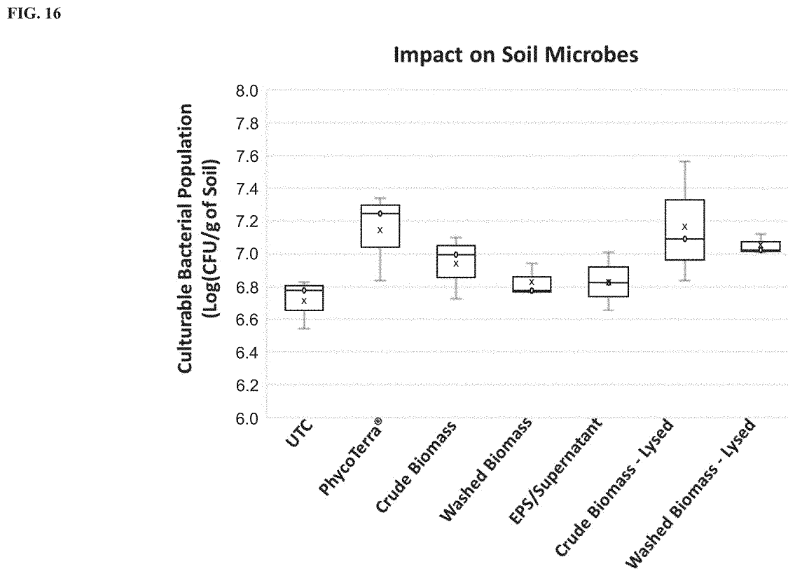

Treatment with microalgal biomass from Parachlorella kessleri Accession No. NCMA 202103001 or with PHYCOTERRA® (whole cell Chlorella microalgae) increased the culturable bacterial populations in the soil samples with the lysed biomass having a more pronounced effect than the whole biomass (see FIG. 16)

### Example 7. Effects of Parachlorella kessleri Accession No. NCMA 202103001 Biomass and EPS on Soil and Soil Microbes

Samples of loam soil from Granger, Iowa and sand soil from Douglas, Ga. were collected and treated with: 1) live-cell crude biomass; 2) live-cell washed biomass; 3) supernatant; 4) lysed crude biomass; 5) lysed washed biomass; 6) PHYCOTERRA® (whole cell Chlorella microalgae) (“PT”); and 7) UTC. Treatments 1) to 5) were each with Parachlorella kessleri Accession No. NCMA 202103001. The supernatant contained EPS separated from cells of Parachlorella kessleri Accession No. NCMA 202103001.

A bacterial community profiling analysis was performed with the treated samples to further investigate the effects of Parachlorella kessleri Accession No. NCMA 202103001 biomass and EPS on microbial populations in different types of soil. Total DNA was extracted from soil samples before subjecting it to PCR to amplify the V3-V4 regions (˜430 bp) of the 16S rRNA gene. The resulting amplicons (i.e., amplified partial 16S rRNA gene) were sequenced using pair-end 2×300 bp Illumina MiSeq platform. The raw 16S rRNA gene sequences from isolated microbial DNA obtained with the different soil types were processed using QIIME2 version 2020.11 (Bolyen et al., 2019). Briefly, single-end reads were imported into QIIME2 and processed with DEBLUR (Amir et al., 2017) to quality filter, trim reads, correct errors, and remove PCR chimeras to obtain representative operational taxonomic unit (OTU) sequences. DEBLUR clustered the resulting sequences at the 100% similarity cutoff and the consensus taxonomy for each OTU was classified using a Naïve Bayes classifier trained on 16S rRNA gene sequences from the SILVA v132 database (Quast et al., 2013).

Statistical analysis of community composition was performed using R version 3.6.1 (R core team, 2020). Bray-Curtis distance was calculated with the R-package phyloseq (McMurdie and Holmes 2013). Bray-Curtis distance was subjected to principal coordinate analysis ordination and significant differences in treatment groups were tested using the ‘adonis’ function in R-package vegan (Oksanen et al., 2020) to implement the PERMANOVA test. Differential abundance testing was carried out using the R-package DESeq2 (Love et al., 2014) to estimate the effective library size and variance to normalize counts prior to detection of pairwise differences between treatments.

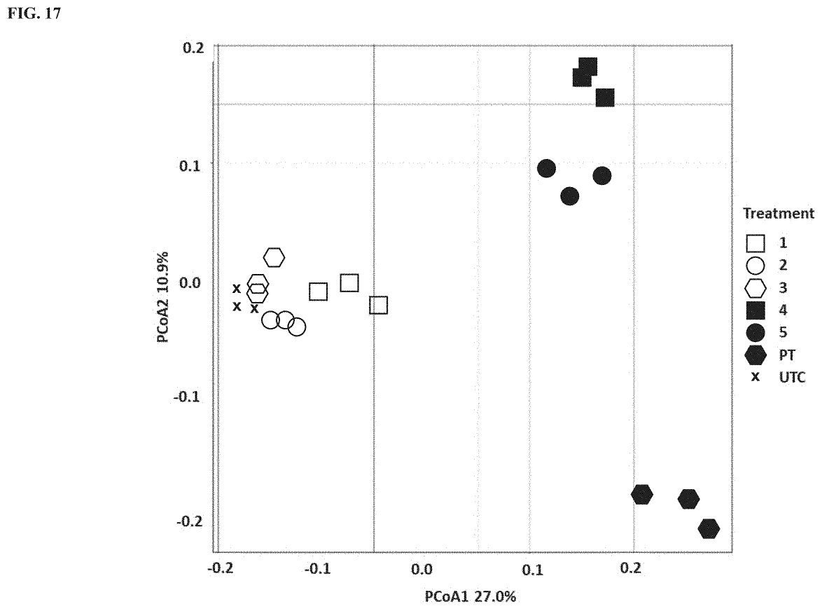

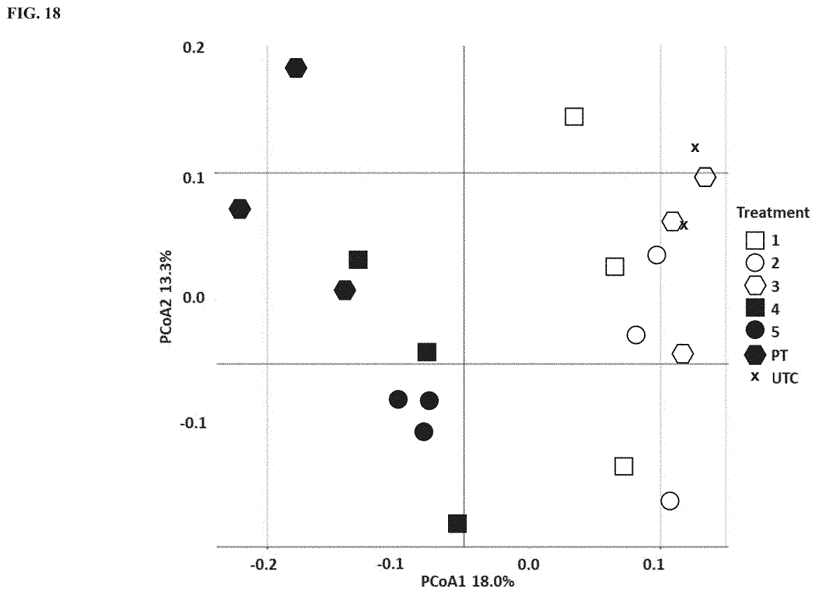

In both the Iowa and Georgia soils, the addition of Parachlorella kessleri Accession No. NCMA 202103001 fractions and PHYCOTERRA® (whole cell Chlorella microalgae) had a significant effect in the microbial community composition (Adonis, p=0.001). Specifically, microbial community composition of soils treated with the live-cell Parachlorella kessleri Accession No. NCMA 202103001 fractions and supernatant clustered with the UTC, while the soil treated with the lysed Parachlorella kessleri Accession No. NCMA 202103001 fractions grouped more closely with the PHYCOTERRA® (whole cell Chlorella microalgae) treated samples (see FIGS. 17-18).

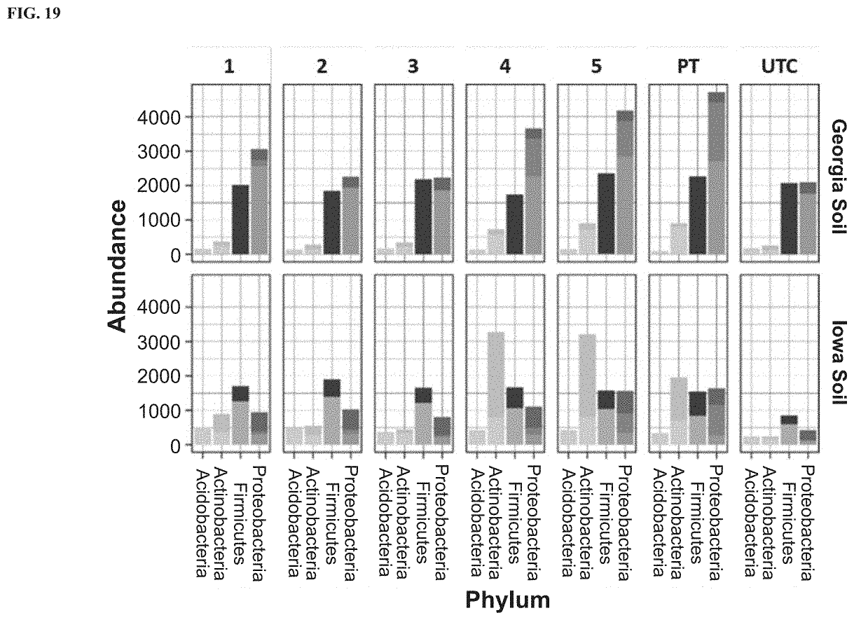

Similarly, with regard to the most abundant taxa, both lysed Parachlorella kessleri Accession No. NCMA 202103001 fractions had a similar effect to PHYCOTERRA® (whole cell Chlorella microalgae) independent of soil type. The addition of PHYCOTERRA® (whole cell Chlorella microalgae) or lysed Parachlorella kessleri Accession No. NCMA 202103001 significantly enriched the proteobacteria in Georgia soils and the actinobacteria in the Iowa soils. In contrast, live-cell fractions and the Parachlorella kessleri Accession No. NCMA 202103001 supernatant treatment had little observable effect on the abundant taxa (see FIG. 19).

### Example 8. Effects of Parachlorella kessleri Accession No. NCMA 202103001 EPS on the Desiccation Resistance of Gram-Negative Bacteria

Rifampicin-resistant strains of two Gram-negative bacteria, Kosakonia pseudosacchari strain C19-Rif and Pseudomonas sp., were mixed with Parachlorella kessleri Accession No. NCMA 202103001 EPS alone or in combination with bacterial EPS in 96-well plates. The liquid medium in the plates was allowed to dry at room temperature either inside a blowing biosafety cabinet (BSC) or a desiccation chamber for several days to create desiccation stress in the bacterial cell cultures. Sterile water containing 0.02% (vol/vol) of a surfactant, SILWET® 77 (alkoxylated trisilane), was then added to the dried 96-well plates, and the resuspended cells were spotted onto culture plates containing rifampicin. The bacterial cells were evaluated for survival and growth after the desiccation stress. A second cycle of drying, resuspension in sterile water, and spotting onto solid medium was performed to evaluate the effect of more severe desiccation stress on the cells.

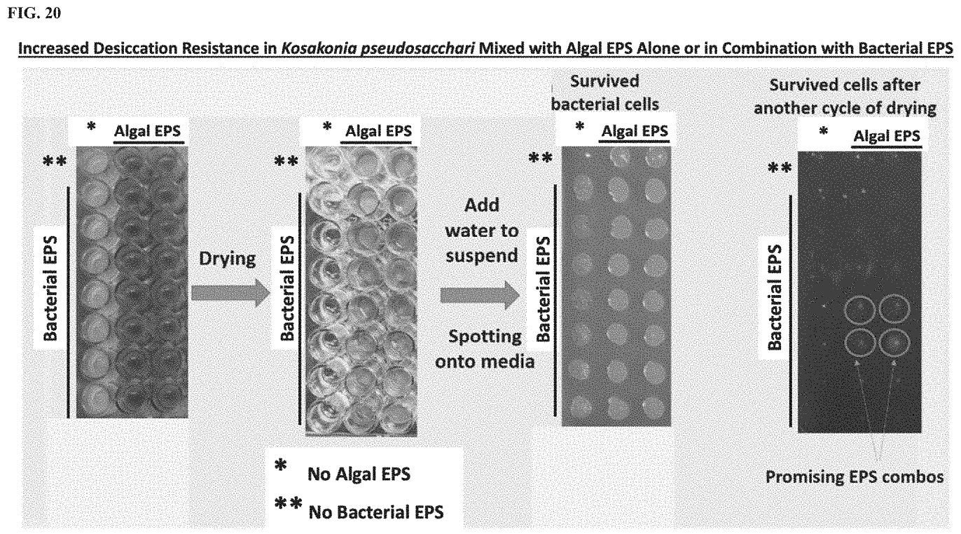

Representative results with the Kosakonia pseudosacchari strain C19-Rif cells are presented in FIG. 20. Similar results were obtained with the Pseudomonas sp. cells. Whereas the Gram-negative bacterial cells without algal EPS or bacterial EPS did not survive after the first round of desiccation stress (see the well in the upper left corner in FIG. 20), Parachlorella kessleri Accession No. NCMA 202103001 crude EPS alone was sufficient to allow the bacterial cells to survive one cycle of desiccation (see the upper middle and right wells in FIG. 20). However, the combination of Parachlorella kessleri Accession No. NCMA 202103001 crude EPS and bacterial EPS was required to preserve the bacterial cells during two cycles of desiccation.

### Example 9. Effects of Porphyridium cruentum UTEX 161 on the Desiccation Resistance of Gram-Negative Bacteria

A rifampicin-resistant strain of a Gram-negative Kosakonia pseudosacchari strain C19-Rif bacterium was mixed with water (untreated control or “UTC”), 60% glycerol (as a stressor), Porphyridium cruentum UTEX 161 whole broth (“Porphy Broth”), Porphyridium cruentum UTEX 161 supernatant (“Porphy Super”), Porphyridium cruentum UTEX 161 pellet (“Porphy Pellet”), Parachlorella kessleri Accession No. NCMA 202103001 whole broth (“3001 Broth”), Parachlorella kessleri Accession No. NCMA 202103001 supernatant (“3001 Super”), or Parachlorella kessleri Accession No. NCMA 202103001 Pellet (“3001 Pellet”) in 96-well plates. Each of the protectant agents was added undiluted (i.e., at a 1× dilution) or at a 2× dilution (i.e., at 2 parts water to 1 part protectant).

The liquid medium in the plates was allowed to dry at room temperature inside a desiccation chamber for several days to create desiccation stress in the bacterial cell cultures. Sterile water containing 0.02% (vol/vol) of a surfactant, SILWET® 77 (alkoxylated trisilane), was then added to the dried 96-well plates, and the resuspended cells were spotted onto culture plates containing rifampicin. The bacterial cells were evaluated for survival and growth after 3 days of desiccation stress and after 5 days of desiccation stress.

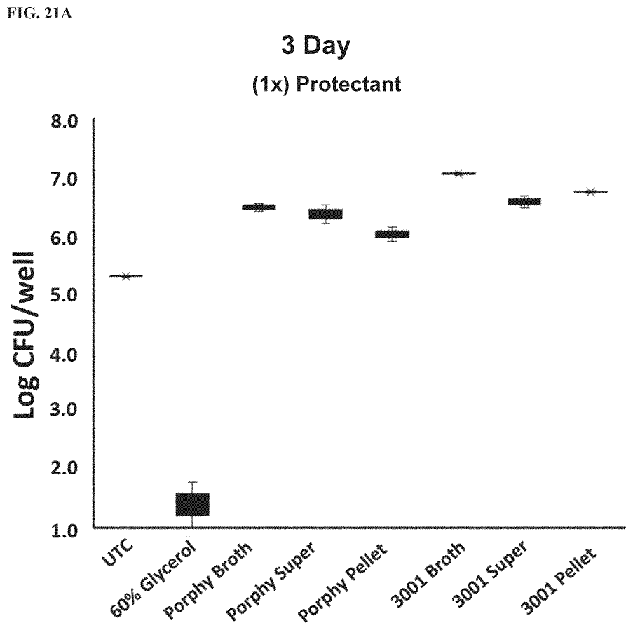

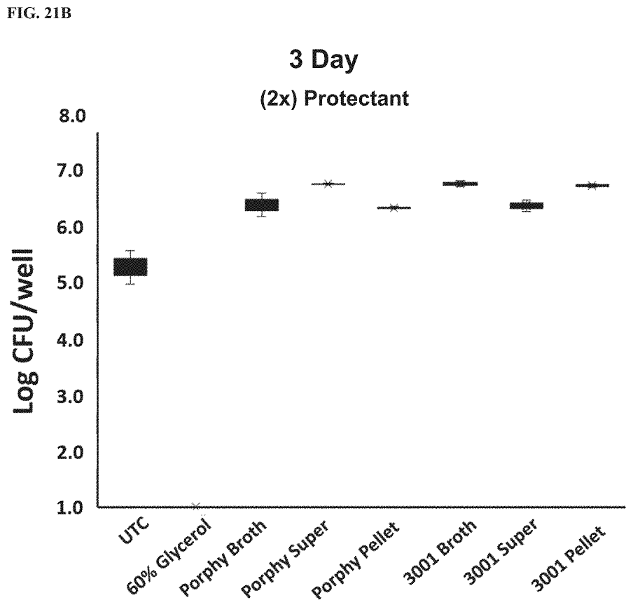

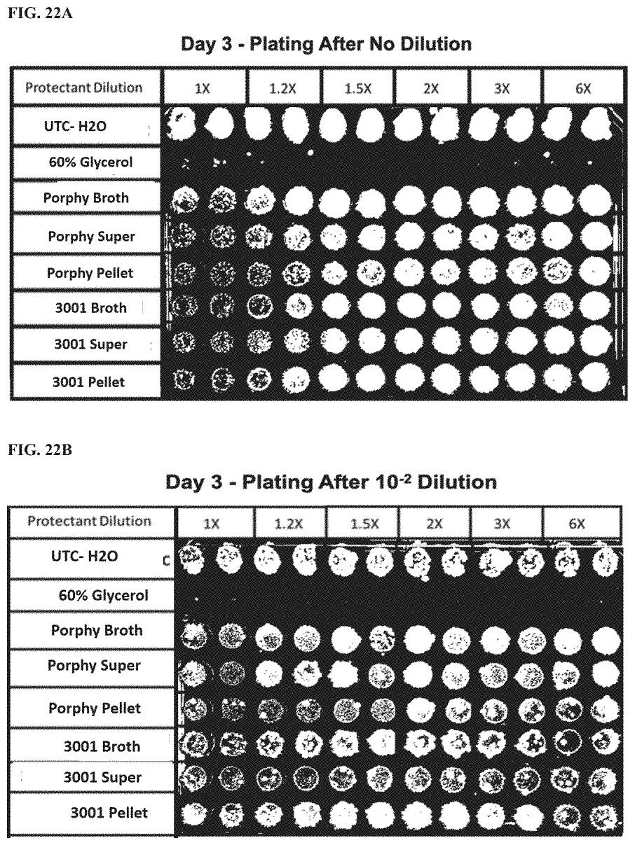

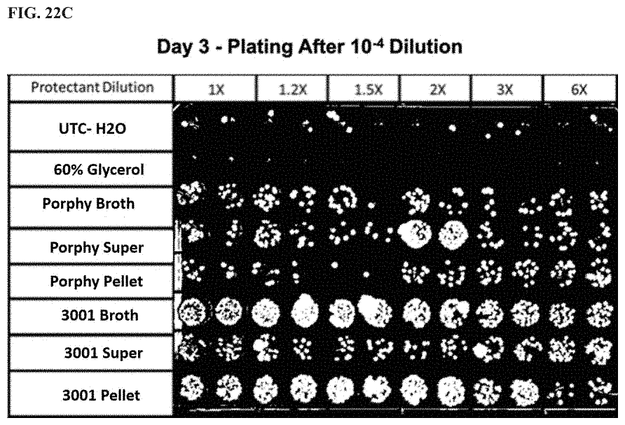

Viability results with the Kosakonia pseudosacchari strain C19-Rif cells after 3 days of desiccation stress are presented in FIGS. 21A and 21B. The colony forming unit (CFU) data in these figures was generated by counting the number of colonies on each spot (see spots on representative plates shown in FIGS. 22A, 22B, and 22C) and using the following formula for quantifying viable cell density:

$$Cell Density ( c f u / mL ) = \frac{\#of Colonies \times Dilution factor}{volume plated ( mL )}$$

In the cases where no colonies were found in the least diluted samples (generally in UTC-H2O samples), the colony number of 0.5 was assigned to calculate cell density and subsequent log transformation.

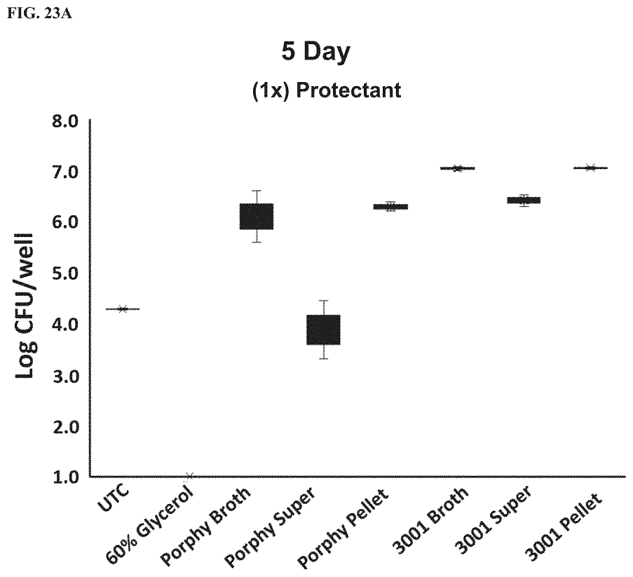

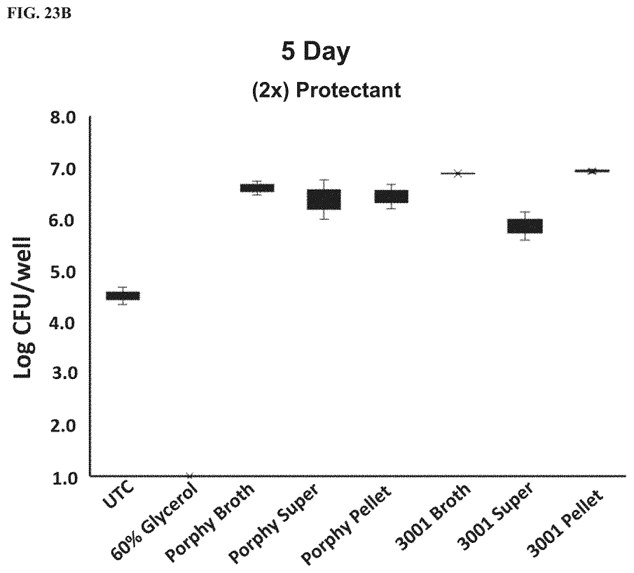

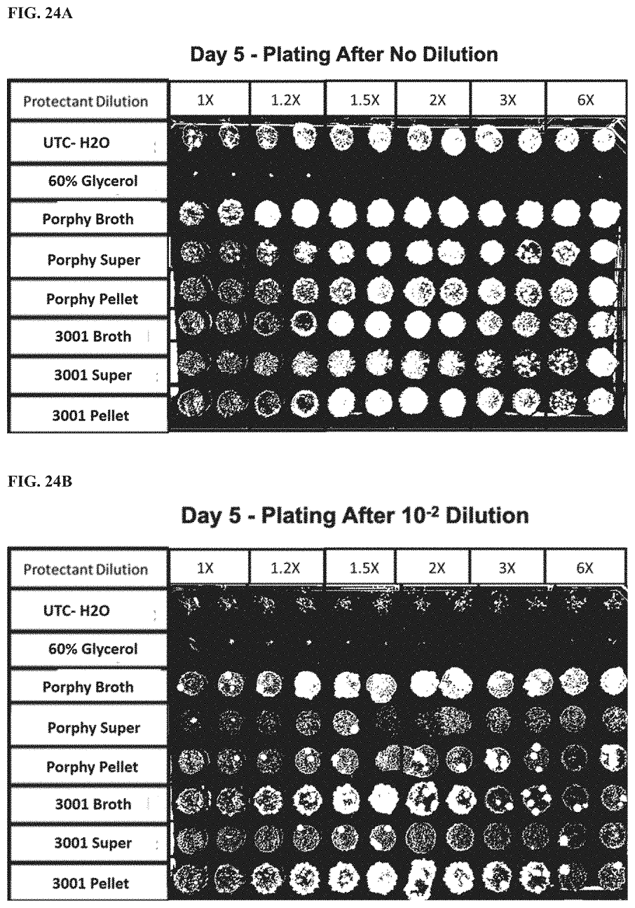

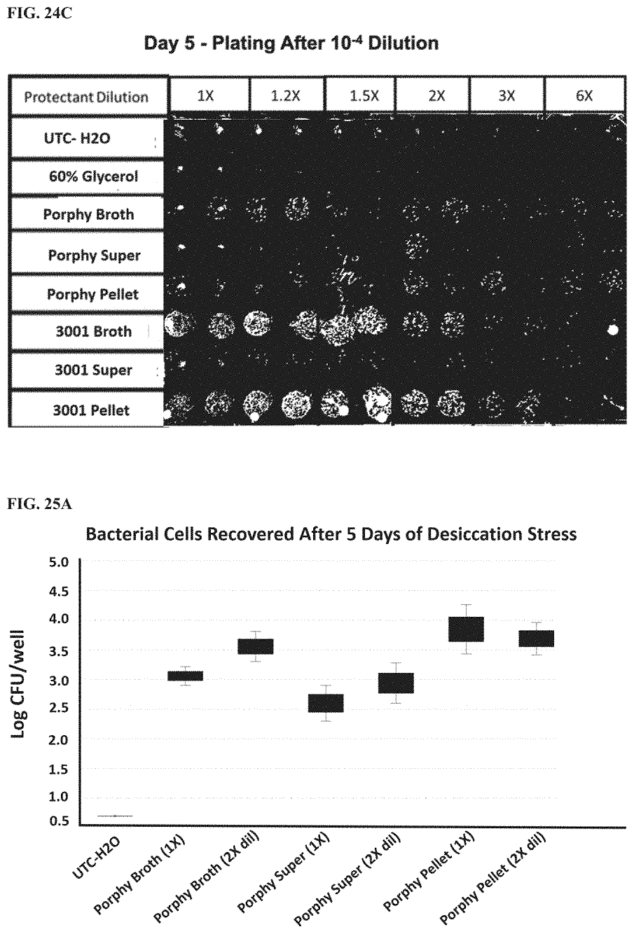

Viability results with the Kosakonia pseudosacchari strain C19-Rif cells after 5 days of desiccation stress are presented in FIGS. 23A and 23B with the corresponding representative plates shown in FIGS. 24A, 24B, and 24C.

The whole broth, supernatant, and cell pellet fractions of Porphyridium cruentum UTEX 161 maintained the viability of the Kosakonia pseudosacchari strain C19-Rif cells during desiccation stress to a similar extent as that observed with the same fractions from Parachlorella kessleri Accession No. NCMA 202103001. Porphyridium cruentum UTEX 161 is a robust producer of EPS, and this EPS separates relatively easily into the supernatant after centrifugation. These results indicate that microalgal EPS from a red algae in the phylum Rhodophyta (i.e., Porphyridium cruentum UTEX 161) as well as microalgal EPS from a green algae in the order Chlorellales (i.e., Parachlorella kessleri Accession No. NCMA 202103001) maintain the viability of Gram-negative bacteria during desiccation stress. In addition, the biomass from these microalgae (e.g., the cell pellet remaining after centrifugation) has a protective effect on Gram-negative bacteria during desiccation stress.

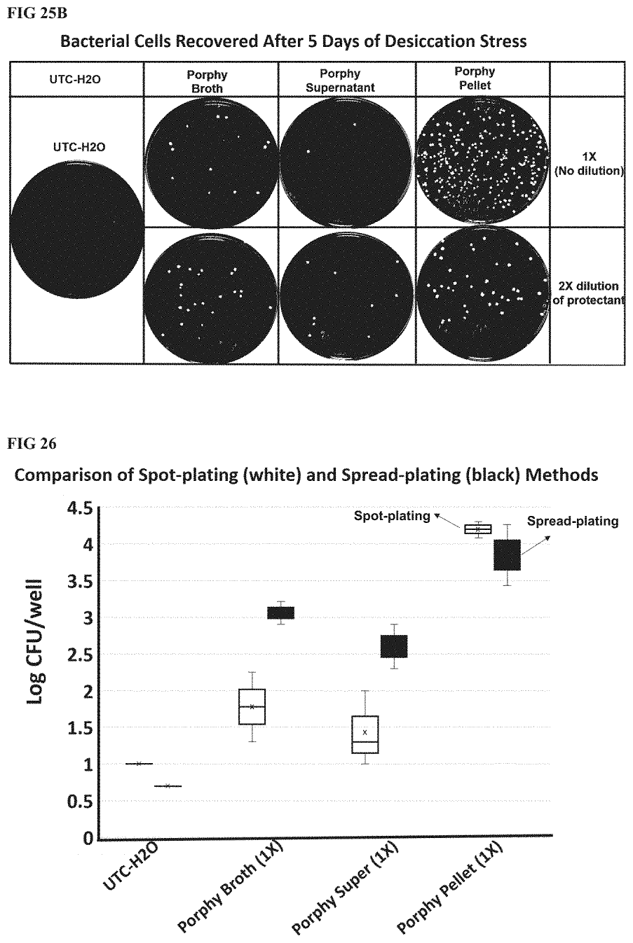

To further quantify the protective effect of different fractions of Porphyridium cruentum toward Kosakonia pseudosacchari strain C19-Rif cells, a separate experiment was performed using a spread-plating method to estimate viable bacterial cells. The spread-plating method used in the experiment affords higher resolution than the spot-plating method but is more labor-intensive than the spot-plating method and requires a large number of agar plates (as each diluent will be plated on one agar plate). In this experiment, a lower initial cell density (1×10⁸ CFU/mL versus 2×10⁹ CFU/mL for the previous experiments in FIGS. 21-24) of Kosakonia pseudosacchari strain C19-Rif was used. Viability results with the Kosakonia pseudosacchari strain C19-Rif cells after 5 days of desiccation stress are presented in FIG. 25A with the corresponding representative plates shown in FIG. 25B. Whereas the undiluted cell pellet fraction of Porphyridium cruentum UTEX 161 provides better desiccation protection than the 2× dilution fraction, the 2× dilution of whole broth and supernatant fractions provides slightly better protection over their undiluted fractions. It might be possible that there is more salt in the broth and supernatant fractions as sea salt (17.5 g/L) was included in the culture broth medium, and this salt could affect the viability of the bacterial cells.

Viability results with the Kosakonia pseudosacchari strain C19-Rif cells were consistent using either the spot-plating method or the spread-plating method, (see the viable cell data in FIG. 26).

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, patents, and patent publications cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

### REFERENCES

Amir A, McDonald D, Navas-Molina J A, Kopylova E, Morton J T, Zech Xu Z, Kightley E P, Thompson L R, Hyde E R, Gonzalez A, Knight R. 2017. Deblur rapidly resolves single-nucleotide community sequence patterns. mSystems 2:e00191-16.
Bolyen, E., Rideout, J. R., Dillon, M. R. et al. Reproducible, interactive, scalable and extensible microbiome data science using QIIME 2. Nat Biotechnol 37, 852-857 (2019).
Dinel H, Levesque P E M, Jambu P, Righi D. 1992. Microbial activity and long-chain aliphatics in the formation of stable soil aggregates. Soil Sci Soc Am J. 1992; 56:1455-1463.
Love M I, Huber W, Anders S (2014). “Moderated estimation of fold change and dispersion for RNA-seq data with DESeq2.” Genome Biology, 15, 550.
McMurdie, P. J. and S. Holmes (2013). “phyloseq: An R Package for Reproducible Interactive Analysis and Graphics of Microbiome Census Data.” PLOS ONE 8(4): e61217.
Oksanen J, Guillaume Blanchet F, Friendly M, Kindt R, Legendre P, McGlinn D, Minchin P R, O'Hara R B, Simpson G L, Solymos P, Henry M, Stevens H, Szoecs E, Wagner H (2020). vegan: Community Ecology Package. R package version 2.5-7.
R Core Team (2020). R: A language and environment for statistical computing. R Foundation for Statistical Computing, Vienna, Austria.
Quast, C., E. Pruesse, P. Yilmaz, J. Gerken, T. Schweer, P. Yarza, J. Peplies and F. O. Glöckner (2013). “The SILVA ribosomal RNA gene database project: improved data processing and web-based tools.” Nucleic Acids Research 41 (Database issue): D590-D596.
Pella M J, Tuomivaara S T, Urbanowicz B R, O'Neill M A, York W S. Methods for structural characterization of the products of cellulose- and xyloglucan-hydrolyzing enzymes. Methods Enzymol. 2012; 510:121-39.
Santander J, Martin T, Loh A, Pohlenz C, Gatlin D M, Curtiss R. Mechanisms of intrinsic resistance to antimicrobial peptides of Edwardsiella ictaluri and its influence on fish gut inflammation and virulence. Microbiology (Reading). 2013 July; 159(Pt 7):1471-1486.
Viji R. and Prasanna P Rajesh. 2012. Assessment of water holding capacity of major soil series of lalgudi, Trichy India. J Environ. Res Develop Vol. 7 No. 1A.
Willis L M, Stupak J, Richards M R, Lowary T L, Li J, Whitfield C. Conserved glycolipid termini in capsular polysaccharides synthesized by ATP-binding cassette transporter-dependent pathways in Gram-negative pathogens. Proc Natl Acad Sci USA. 2013 May 7; 110(19):7868-73.

What is claimed is:

1. A composition comprising:
an extracellular polymeric substance produced by microalgae;
plant growth promoting Gram-negative bacteria; and
an agriculturally acceptable carrier.

2. The composition of claim 1, further comprising biomass produced by the microalgae or a fraction thereof.

3. The composition of claim 2, wherein the biomass is a whole broth culture or a cell pellet of the microalgae.

4. (canceled)

5. The composition of claim 1, wherein the microalgae are green algae in the order Chlorellales.

6. (canceled)

7. The composition of claim 5, wherein the green algae belong to the genus Parachlorella.

8. (canceled)

9. The composition of claim 1, wherein the microalgae are red algae in the phylum Rhodophyta.

10. (canceled)

11. The composition of claim 9, wherein the red algae belong to the genus Porphyridium.

12. (canceled)

13. The composition of claim 1, wherein the extracellular polymeric substance comprises an exopolysaccharide.

14. (canceled)

15. (canceled)

16. (canceled)

17. A plant propagation material treated with the composition of claim 1.

18. The plant propagation material of claim 17, wherein the plant propagation material is a seed.

19. A method of increasing resistance to desiccation in plant growth promoting Gram-negative bacteria, the method comprising adding an extracellular polymeric substance produced by microalgae to the plant growth promoting Gram-negative bacteria.

20. The method of claim 19, further comprising adding biomass produced by the microalgae or a fraction thereof to the plant growth promoting Gram-negative bacteria.

21. The method of claim 20, wherein the biomass is a whole broth culture or a cell pellet of the microalgae.

22. (canceled)

23. (canceled)

24. The method of claim 19, wherein the microalgae are green algae belonging to the genus Parachlorella.

25. (canceled)

26. (canceled)

27. The method of claim 19, wherein the microalgae are red algae belonging to the genus Porphyridium.

28. (canceled)

29. The method of claim 19, wherein the extracellular polymeric substance comprises an exopolysaccharide.

30. (canceled)

31. (canceled)

32. An isolated biologically pure culture of Parachlorella kessleri Accession No. NCMA 202103001 or a mutant thereof having all the identifying characteristics thereof.

33. A cell-free preparation or an extracellular polymeric substance of the isolated biologically pure culture of Parachlorella kessleri Accession No. NCMA 202103001 or mutant thereof of claim 32.

34. (canceled)

35. (canceled)

36. A seed treatment comprising a composition comprising the biologically pure culture of claim 32.

37. (canceled)

38. (canceled)

39. A seed treatment comprising a composition comprising a cell-free preparation or an extracellular polymeric substance of the isolated biologically pure culture of Parachlorella kessleri Accession No. NCMA 202103001 or mutant thereof of claim 32.


SEQUENCE LISTING (4 sequences)

SEQ ID NO: 1 - DNA, 21 bp, synthetic construct
  misc_feature 1..21: note = Primer
  source 1..21: mol_type = other DNA
aacctggttgatcctgccagt

SEQ ID NO: 2 - DNA, 16 bp, synthetic construct
  misc_feature 1..16: note = Primer
  source 1..16: mol_type = other DNA
gggcatcacagacctg

SEQ ID NO: 3 - DNA, 19 bp, synthetic construct
  misc_feature 1..19: note = Primer
  source 1..19: mol_type = other DNA
tccgtaggtgaacctgcgg

SEQ ID NO: 4 - DNA, 20 bp, synthetic construct
  misc_feature 1..20: note = Primer
  source 1..20: mol_type = other DNA
tcctccgcttattgatatgc